(19) United States
(12) Patent Application Publication
(10) Pub. No.: US 2024/0002598 A1
(43) Pub. Date: Jan. 4, 2024

(54) POLYMERIC PRODUCTS FORMED USING POLYBENZOXAZINES SUITABLE FOR USE IN ADDITIVE MANUFACTURING

(71) Applicant: Nanyang Technological University, Singapore (SG)

(72) Inventors: Xiao HU, Singapore (SG); Yong LU, Singapore (SG); Kok Wei Joseph NG, Singapore (SG); Hui CHEN, Singapore (SG)

(21) Appl. No.: 18/252,093

(22) PCT Filed: Nov. 10, 2021
(86) PCT No.: PCT/SG2021/050686

§ 371 Date: May 8, 2023

(30) Foreign Application Priority Data
Nov. 10, 2020 (SG) 10202011174V

(51) Int. Cl. C08G 73/02; C07D 265/16; C08J 3/28; C08F 220/36; C08J 3/24; B29C 64/30; B33Y 10/00; B33Y 40/20; B33Y 70/00

(52) U.S. Cl. CPC C08G 73/0233; C07D 265/16; C08J 3/28; C08F 220/36; C08J 3/24; B29C 64/30; B33Y 10/00; B33Y 40/20; B33Y 70/00; C08J 2333/14; C08J 2433/08; B29K 2077/00

(57) ABSTRACT

Disclosed herein is a polymeric product formed from a cured polymeric material that comprises a repeating unit derived from a monomer according to formula I or formula II: where Ra, Rb and R1 to R6 are defined herein. Also disclosed herein are the monomers according to formula I and II, as well as formulations comprising said monomers.

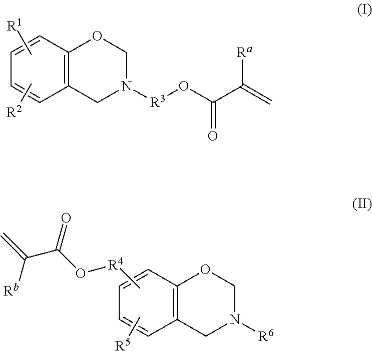

### FIELD OF INVENTION

The current invention relates to a polymeric product that may be fully or partly cured that is formed using a polybenzoxazine derivative, to the polybenzoxazine derivatives themselves and their use in additive manufacturing.

### BACKGROUND

The listing or discussion of a prior-published document in this specification should not necessarily be taken as an acknowledgement that the document is part of the state of the art or is common general knowledge.

Polybenzoxazines (PBZs) are a class of high-performance thermosetting phenolics which have demonstrated a range of desirable features to overcome some of the limitations of conventional novolac and resole type phenolics (C. P. R. Nair, Prog. Polym. Sci. 2004, 29, 401-498; N. N. Ghosh, B. Kiskan & Y. Yagci, Prog. Polym. Sci. 2007, 32, 1344-1391; and S. Wirasate et al., J. Appl. Polym. Sci. 1998, 70, 1299-1306). Thermosetting PBZs are prepared by thermal treating benzoxazine (BZ) monomers. PBZs offer a variety of advantages such as high thermal stability and mechanical strength, high char yield, excellent flame resistance, low water absorption and near-zero volumetric shrinkage (Y. Yagci, B. Kiskan & N. N. J. Ghosh, J. Polym. Sci., Part A: Polym. Chem. 2009, 47, 5565-5576; Y. X. Wang & H. Ishida, J. Appl. Polym. Sci. 2002, 86, 2953-2966; H. D. Kim & H. Ishida, Macromolecules 2003, 36, 8320-8329; and L. Dumas et al., Chem. Commun. 2013, 49, 9543-9545). However, there are some inherent shortcomings that PBZs have. For instance, they have brittle natures and undesirable processability due to high curing temperatures (generally 180-250° C.) required. Therefore, the use of conventional manufacturing methods such as extrusion and melting to process PBZs into complicated structures are difficult, and this limits their wide implementation. Additive manufacturing (AM), commonly known as 3D printing, is a rapidly developing technology that has advanced product fabrication in prototyping and tooling, and offers a revolutionary alternative for material processing away from traditional manufacturing methods, with the major advantage of accurately producing complex structures and shapes (B. Narupai & A. Nelson, ACS Macro Lett. 2020, 9, 627-638; and S. C. Ligon et al., Chem. Rev. 2017, 117, 10212-10290).

Therefore, there is a need to discover new formulations of photoprintable resins for the efficient fabrication of high-performance PBZ thermosets via AM, for various engineering applications.

### SUMMARY OF INVENTION

Aspects and embodiments of the invention will now be discussed by reference to the following numbered clauses.

1. A polymeric product, wherein the polymeric product is formed from a cured polymeric material that comprises a repeating unit derived from a monomer according to formula I or formula II:

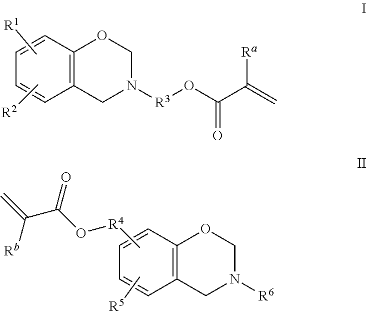

where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
wherein the polymeric product has been subjected to photocuring followed by thermal curing.

2. A polymeric product, wherein the polymeric product is formed from a partly-cured polymeric material that comprises a repeating unit derived from a monomer according to formula I or formula II:

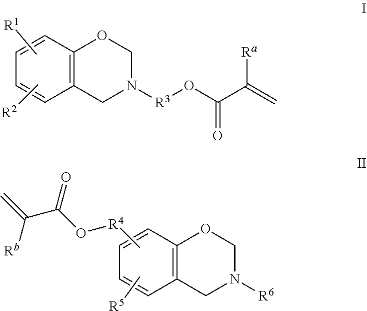

each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
wherein the polymeric product has been subjected to photocuring.

3. The polymeric product according to Clause 1 or Clause 2, wherein the monomer has a viscosity of less than 5 Pa/s, such as less than 2.5 Pa/s, such as less than 0.9 Pa/s, when measured as a neat monomer.

4. A monomer according to formula I or formula II:

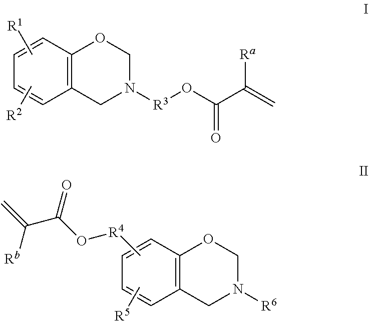

where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
provided that when the monomer is a compound of formula I where R³ is —CH₂CH₂—, then one of R¹ and R² is not H.

5. The monomer according to Clause 4, wherein the monomer has a viscosity of less than 5 Pa/s, such as less than 2.5 Pa/s, such as less than 0.9 Pa/s, when measured as a neat monomer.

6. The monomer according to Clause 4 or Clause 5, wherein the monomer according to formula I or formula II is a monomer according to formula Ia or formula IIa:

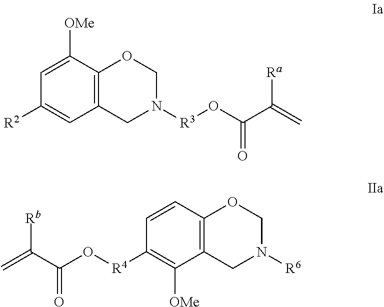

where:
R² is selected from H, CO₂R⁷. linear or branched C₁ to C₅ alkyl, linear or branched C₂ to C₅ alkenyl, where the latter two substituents are unsubstituted or substituted by one or more substituents selected from OR^{8a}, ═O, and C₁ to C₃ alkyl;
R³ is a linear or branched C₂ to C₅ alkyl;
R⁴ is a linear or branched C₁ to C₅ alkyl;
R⁶ is a linear or branched C₁ to C₅ alkyl, which is unsubstituted or substituted by one or more substituents selected from OR^{8a}, ═O, and C₁ to C₃ alkyl;
R⁷ is a linear or branched C₁ to C₅ alkyl; and
R^{a} and R^{b} are selected from H and CH₃.

7. The monomer according to Clause 6, wherein:
R² is selected from H, CO₂R⁷. linear or branched C₂ to C₄ alkyl, linear or branched C₂ to C₃ alkenyl, where the latter two substituents are unsubstituted or substituted by one or more substituents selected from ═O, and C₁ to C₃ alkyl;
R³ is a linear or branched C₂ to C₅ alkyl;
R⁴ is C₁ alkyl;
R⁶ is a linear or branched C₂ to C₄ alkyl, which is unsubstituted or substituted by one or more substituents selected from C₁ to C₃ alkyl;
R⁷ is a linear or branched C₂ to C₄ alkyl; and
R^{a} and R^{b} are selected from H and OH₃.

8. A monomer selected from the list:

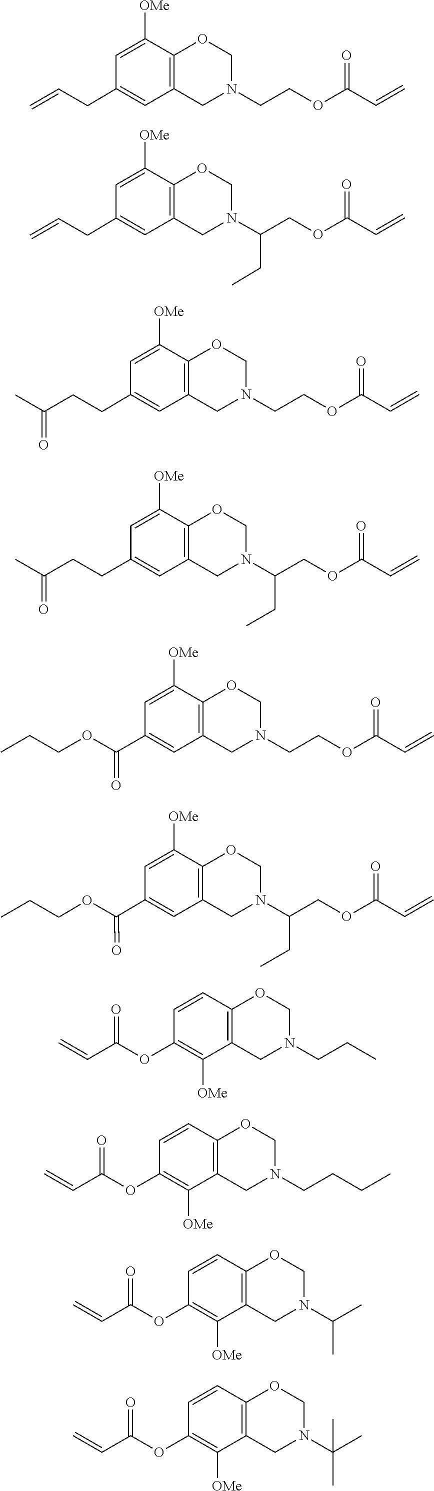

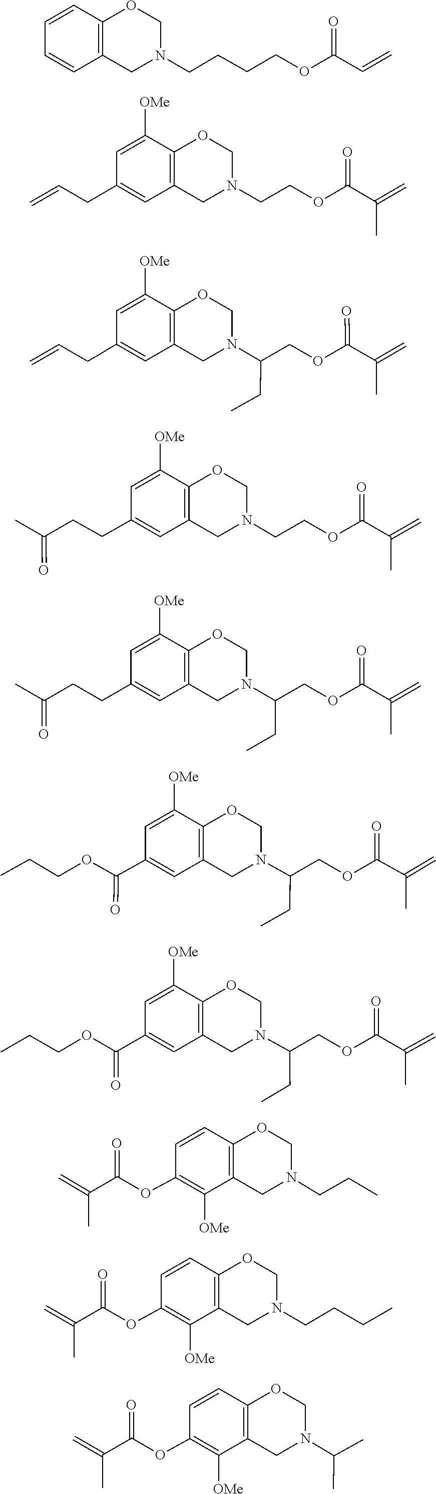

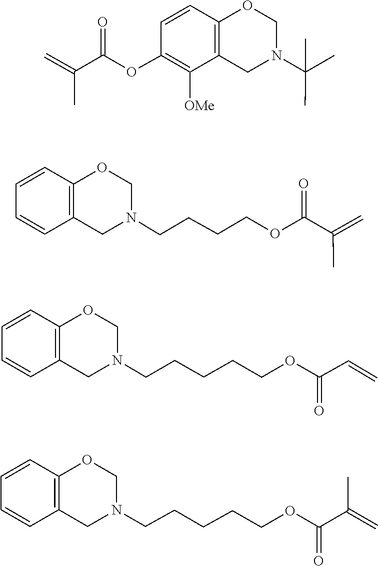 [SMILES: C=C(C)C(=O)Oc1ccc2c(c1OC)CN(C(C)(C)C)CO2 ; C=C(C)C(=O)OCCCCN1COc2ccccc2C1 ; C=CC(=O)OCCCCCN1COc2ccccc2C1 ; C=C(C)C(=O)OCCCCCN1COc2ccccc2C1]

9. A formulation for additive manufacturing, comprising:
one or more monomer according to formula I or formula II:

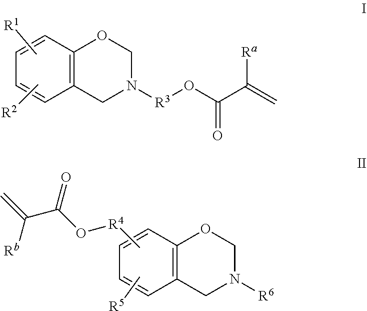

and a photoinitiator;
where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl.

10. The formulation according to Clause 9, wherein:
(ai) when the monomer is a compound of formula I where R³ is —CH₂CH₂—, then one of R¹ and R² is not H; and/or
(aii) the monomer has a viscosity of less than 5 Pa/s, such as less than 2.5 Pa/s, such as less than 0.9 Pa/s, when measured as a neat monomer.

11. The formulation according to Clause 9 or Clause 10, wherein the formulation further comprises one or more of an antioxidant, a stabiliser, a colourant, a diluent, a flame retardant, a plasticizer, a photoabsorber, a photoinhibitor, and a filler.

12. The formulation according to any one of Clauses 9 to 11, wherein the formulation further comprises an acrylate monomer that does not have the formula I or the formula II.

13. The formulation according to any one of Clauses 9 to 12, wherein the monomer according to formula I or formula II is a monomer according to formula Ia or formula IIa:

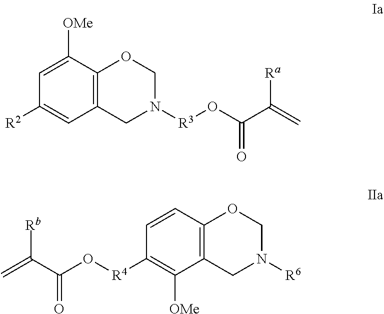

where:
R² is selected from H, CO₂R⁷. linear or branched C₁ to C₅ alkyl, linear or branched C₂ to C₅ alkenyl, where the latter two substituents are unsubstituted or substituted by one or more substituents selected from OR^{8a}, ═O, and C₁ to C₃ alkyl;
R³ is a linear or branched C₂ to C₅ alkyl;
R⁴ is a linear or branched C₁ to C₅ alkyl;
R⁶ is a linear or branched C₁ to C₅ alkyl, which is unsubstituted or substituted by one or more substituents selected from OR^{8a}, ═O, and C₁ to C₃ alkyl;
R⁷ is a linear or branched C₁ to C₅ alkyl; and
R^{a} and R^{b} are selected from H and CH₃.

14. The formulation according to Clause 13, wherein:
R² is selected from H, CO₂R⁷. linear or branched C₂ to C₄ alkyl, linear or branched C₂ to C₃ alkenyl, where the latter two substituents are unsubstituted or substituted by one or more substituents selected from ═O, and C₁ to C₃ alkyl;
R³ is a linear or branched C₂ to C₅ alkyl;
R⁴ is C₁ alkyl;
R⁶ is a linear or branched C₂ to C₄ alkyl, which is unsubstituted or substituted by one or more substituents selected from C₁ to C₃ alkyl;
R⁷ is a linear or branched C₂ to Ca alkyl.

15. The formulation according to any one of Clauses 9 and 11 to 14, wherein the monomer is selected from the list:

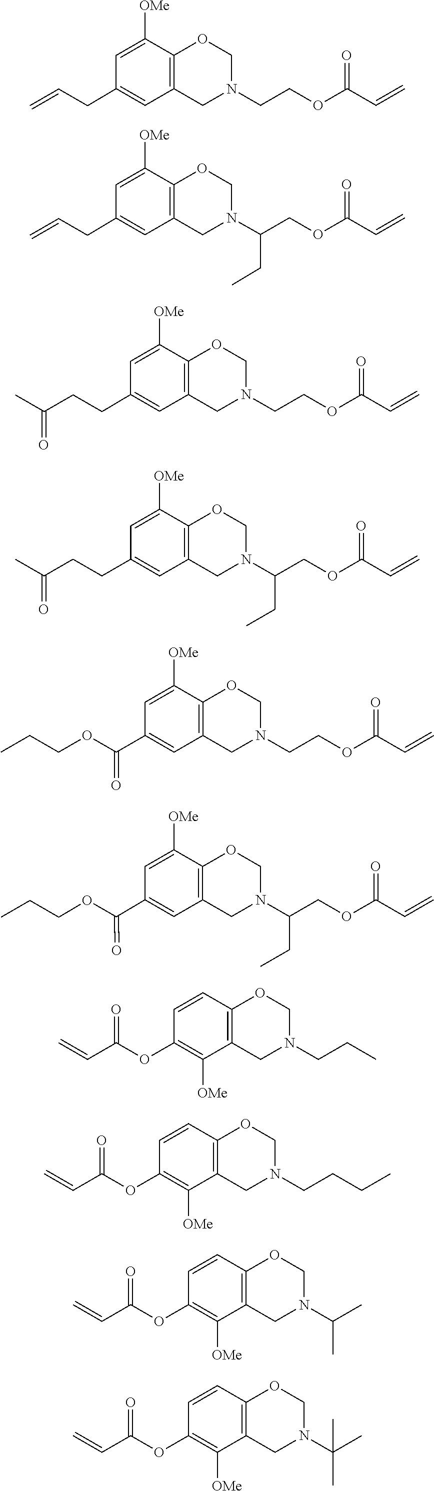

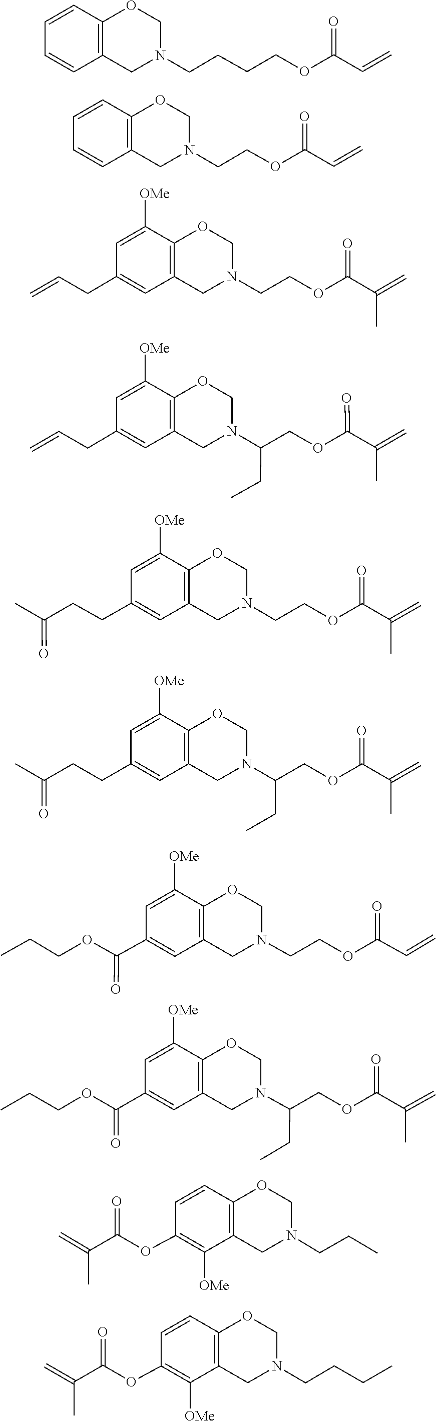

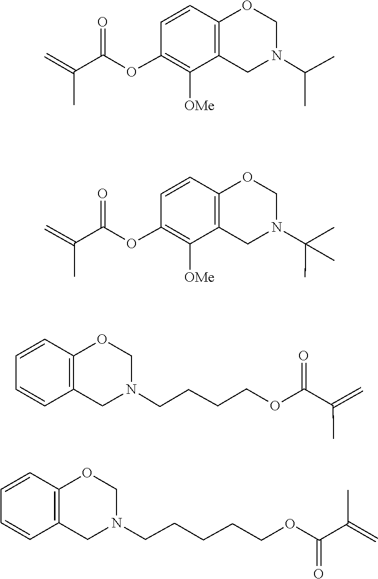

16. The formulation according to any one of Clauses 9 to 15, wherein the monomer is selected from the list:

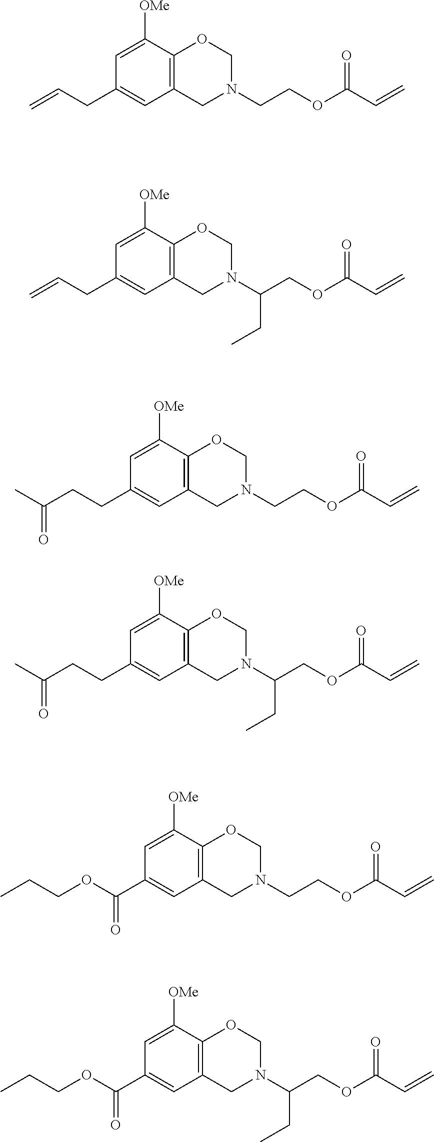

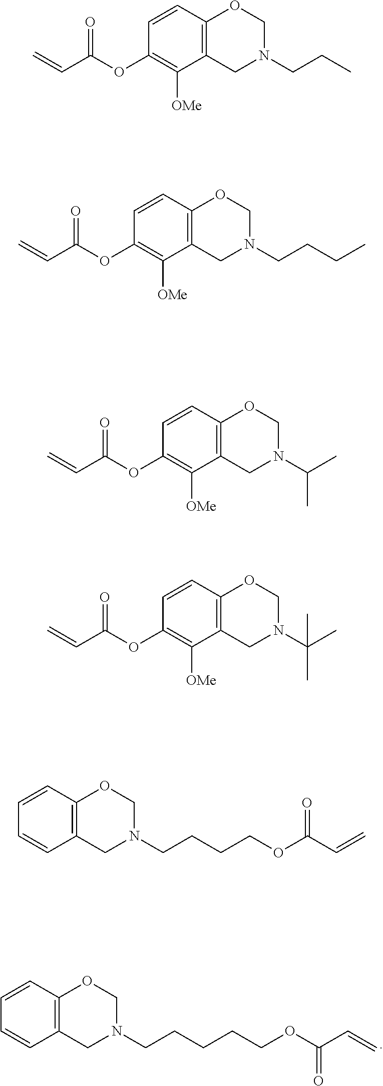

17. The formulation according to any one of Clauses 9 to 16, wherein the formulation comprises the following monomers:

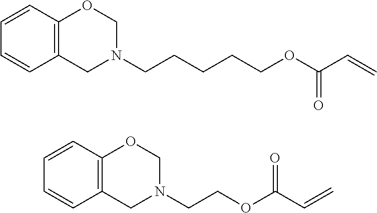 [SMILES: C=CC(=O)OCCCCCN1COc2ccccc2C1 ; C=CC(=O)OCCN1COc2ccccc2C1]

18. A method of providing an intermediate product by additive manufacturing, the method comprising the steps of:
(a) 3D-printing an object one layer at a time according to a product design using a printing resin, where each layer is subjected to ultraviolet light for a first period of time before each further layer is added; and
(b) stopping the 3D-printing once the product design has been generated, thereby providing an intermediate product, wherein:
   the printing resin is either a monomer according to any one of Clauses 4 to 8 or a formulation according to any one of Clauses 9 to 17; and
   the product is capable of being cured further using a thermal curing step.

19. The method according to Clause 18, wherein the intermediate product is subjected to ultraviolet light for a second period of time to provide a further intermediate product.

20. The method according to Clause 18 or Clause 19, wherein the method further comprises a step of providing a final product by subjecting the intermediate product to a thermal curing step using a suitable temperature for a third period of time.

21. A method of providing a final product by additive manufacturing, the method comprising the steps of:
(a) a providing an intermediate product formed by an additive manufacturing process; and
(b) subjecting the intermediate product to a thermal curing step using a suitable temperature for a period of time, wherein
   the intermediate product is formed from polymeric matrix material, where the polymeric matrix material comprises repeating units derived from a monomer according to any one of Clauses 4 to 8.

22. The method according to Clause 21, wherein the intermediate product is formed by a method comprising the steps of:
(a) 3D-printing an object one layer at a time according to a product design using a printing resin, where each layer is subjected to ultraviolet light for a first period of time before each further layer is added; and
(b) stopping the 3D-printing once the product design has been generated, thereby providing an intermediate product, wherein:
   the printing resin is either a monomer according to any one of Clauses 4 to 8 or a formulation according to any one of Clauses 9 to 17; and
   the product is capable of being cured further using a thermal curing step.

23. The method according to Clause 22, wherein the intermediate product is subjected to ultraviolet light for a second period of time to provide a further intermediate product.

24. The method according to Clause 22 or Clause 23, wherein the method further comprises a step of providing a final product by subjecting the intermediate product to a thermal curing step using a suitable temperature for a third period of time.

### DRAWINGS

FIG. 1 depicts (a) synthetic routes; (b) plot of viscosity vs shear rate of BZ-C2, BZ-C5 and BZ-BA; (c) UV-vis absorption of BZ-C2 and BZ-C5 in dilute chloroform solution; (d) differential scanning calorimetry (DSC) thermograms for PBZ-C2 cured at different temperature; and (e) thermogravimetric analysis (TGA) curves of photocured BZ-C2/C5 and PBZ-C2/C5 in N₂ atmosphere.

FIG. 2 depicts the Fourier transform infrared (FT-IR) spectra of monomers, photo cured BZ-C2/C5 and PBZ-C2/C5.

FIG. 3 depicts the TGA curve of a commercial resin (GR20) in N₂ atmosphere.

FIG. 4 depicts (a) storage modulus and (b) tan δ plotted as a function of temperature for photocured BZ-C2/C5 and PBZ-C2/C5; (c) flexure stress-strain curves of PBZ-C2 and PBZ-C5 thermally cured under different temperatures and (d) their tabulated mechanical performance data; (e) schematic illustration of the triple networks formed in the PBZs; and (f) 30 photographs of (i) BZ-C2; and (ii) BZ-C5 before and after thermal curing.

FIG. 5 depicts a general ring opening reaction mechanism and the corresponding network structures of UV cured BZ and PBZ.

FIG. 6 depicts T_{g} and modulus of PBZ-C2 and PBZ-C5 compared with literature results. ▪: 1—Acrylate resins; •: 2—Epoxy resins; ▴: 3—Hybrid resins; ▾: 4—Phenolic resins; ♦: 5—Cyanate ester resins; and [symbol] : 6—This work.

FIG. 7 depicts (a) schematic illustration of PμSL printing process; (b) height changes and 3D printed diverse structures before (top) and after (bottom) thermal treatment; (i) honeycomb (BZ-C5 based resin); (ii) gear (BZ-C2 based resin); and (iii) component (BZ-C2 based resin). Scale bar: 1 mm; and c) Scanning electron microscopy (SEM) images of the fracture surface of the printed NTU logo (BZ-C2 based resin) before and after thermal treatment.

FIG. 8 depicts photocurable behaviour and printability of (a) BZ-C2:BC3(20%/30%); (b) BZ-C5:BC3(20%/30%); and (c) printed patterns of BZ-C2:BC3(30%).

### DESCRIPTION

In a first aspect of the invention, there is provided a polymeric product, wherein the polymeric product is formed from a cured polymeric material that comprises a repeating unit derived from a monomer according to formula I or formula II:

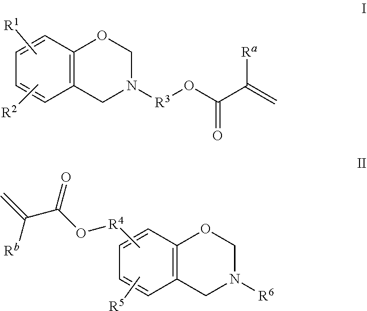

where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8e}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
wherein the polymeric product has been subjected to photocuring followed by thermal curing.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

It is believed that a polymeric product that has been subjected to photocuring followed by thermal curing will be a material where substantially all of the carbon-to-carbon double bonds available for reaction have been reacted together (i.e. the product is a substantially fully cured product). It is believed that if only thermal curing (or photocuring) are used individually, then only some of the carbon-to-carbon double bonds available for reaction will have been reacted, leaving a proportion of unreacted double bonds (e.g. 5% or more, such as 10% or more). Therefore, the product of this aspect of the invention is believed to be chemically and physically distinct from a partly-cured product.

When used herein, the term “substantially all of the carbon-to-carbon double bonds available for reaction have been reacted together” means that less than 5%, such as less than 4%, such as less than 3%, such as less than 2%, such as less than 1%, such as less than 0.5%, such as less than 0.1%, such as less than 0.01%, such as none of the carbon-to-carbon double bonds available for reaction remain in the product.

The term “halo”, when used herein, includes references to fluoro, chloro, bromo and iodo.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, acyclic or cyclic, saturated or unsaturated (so forming, for example, an alkenyl or alkynyl)hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

The term “heteroaryl” when used herein refers to an aromatic group containing one or more heteroatom(s) (e.g. one to four heteroatoms) preferably selected from N, O and S (so forming, for example, a mono-, bi-, or tricyclic heteroaromatic group). Heteroaryl groups include those which have between 5 and 14 (e.g. 10) members and may be monocyclic, bicyclic or tricyclic, provided that at least one of the rings is aromatic. However, when heteroaryl groups are bicyclic or tricyclic, they are linked to the rest of the molecule via an aromatic ring. Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pteridinyl. Particularly preferred heteroaryl groups include monocyclic heteroaryl groups.

Unless otherwise specified herein, a “heterocyclic ring system” may be 4- to 14-membered, such as a 5- to 10-membered (e.g. 6- to 10-membered), heterocyclic group that may be aromatic, fully saturated or partially unsaturated, and which contains one or more heteroatoms selected from O, S and N, which heterocyclic group may comprise one or two rings. Examples of heterocyclic ring systems that may be mentioned herein include, but are not limited to azetidinyl, dihydrofuranyl (e.g. 2,3-dihydrofuranyl, 2,5-dihydrofuranyl), dihydropyranyl (e.g. 3,4-dihydropyranyl, 3,6-dihydropyranyl), 4,5-dihydro-1H-maleimido, dioxanyl, dioxolanyl, furanyl, furazanyl, hexahydropyrimidinyl, hydantoinyl, imidazolyl, isothiaziolyl, isoxazolidinyl, isoxazolyl, morpholinyl, 1,2- or 1,3-oxazinanyl, oxazolidinyl, oxazolyl, piperidinyl, piperazinyl, pyranyl, pyrazinyl, pyridazinyl, pyrazolyl, pyridinyl, pyrimidinyl, pyrrolinyl (e.g. 3-pyrrolinyl), pyrrolyl, pyrrolidinyl, pyrrolidinonyl, 3-sulfolenyl, sulfolanyl, tetrahydrofuranyl, tetrahydropyranyl, tetrahydropyridinyl (e.g. 3,4,5,6-tetrahydropyridinyl), 1,2,3,4-tetrahydropyrimidinyl, 3,4,5,6-tetrahydropyrimidinyl, tetrahydrothiophenyl, tetramethylenesulfoxide, tetrazolyl, thiadiazolyl, thiazolyl, thiazolidinyl, thienyl, thiophenethyl, triazolyl and triazinanyl.

Unless otherwise specified herein, a “carbocyclic ring system” may be 4- to 14-membered, such as a 5- to 10-membered (e.g. 6- to 10-membered, such as a 6-membered or 10-membered), carbocyclic group that may be aromatic, fully saturated or partially unsaturated, which carbocyclic group may comprise one or two rings. Examples of carbocyclic ring systems that may be mentioned herein include, but are not limited to cyclobutyl, cyclopentyl, cyclohexyl, cyclooctyl, phenyl, naphthyl, decalinyl, tetralinyl, bicyclo[4.2.0]octanyl, and 2,3,3a,4,5,6,7,7a-octahydro-1H-indanyl. Particularly preferred carbocyclic groups include phenyl, cyclohexyl and naphthyl.

In a second aspect of the invention, there is disclosed a polymeric product, wherein the polymeric product is formed from a partly-cured polymeric material that comprises a repeating unit derived from a monomer according to formula I or formula II:

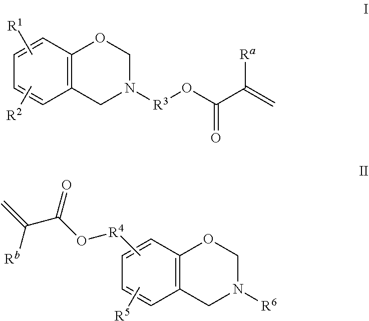

where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
wherein the polymeric product has been subjected to photocuring.

It will be appreciated that the terms defined hereinbefore also apply to this aspect of the invention too. Furthermore, it will be appreciated that a partly cured polymeric product that has been subjected to photocuring will look different (and may have different chemical and physical properties) to one that has undergone thermal curing. As will be appreciated, a partly-cured polymeric product as mentioned herein may be an intermediate product that may itself be prepared, stored and subjected to a final thermal curing process to provide the desired final product (fully cured) at a different time.

In a third aspect of the invention, there is provided a monomer according to formula I or formula II:

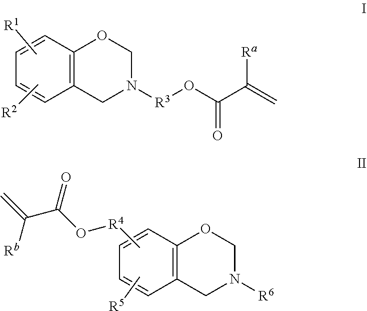

where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
provided that when the monomer is a compound of formula I where R³ is —CH₂CH₂—, then one of R¹ and R² is not H.

As will be appreciated, the same terms used in this aspect adopts the same definitions as provided above.

In the first, second and third aspects of the invention, the monomer may have any suitable viscosity. However, it may be advantageous for the monomer (when measured as the neat monomer) to be one that has a viscosity that is less than 5 Pa/s, such as less than 2.5 Pa/s, such as less than 0.9 Pa/s. This may mean that the monomer can be used without the need to include any diluents or other additives to reduce the viscosity of the monomer for use in additive manufacturing.

In the first to third aspects that have been mentioned herein, the monomer according to formula I or formula II may be a monomer according to formula Ia or formula IIa:

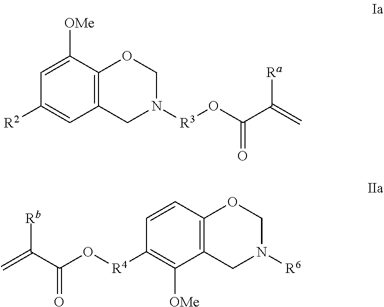

where:
R² is selected from H, CO₂R⁷. linear or branched C₁ to C₅ alkyl, linear or branched C₂ to C₅ alkenyl, where the latter two substituents are unsubstituted or substituted by one or more substituents selected from OR^{8a}, ═O, and C₁ to C₃ alkyl;
R³ is a linear or branched C₂ to C₅ alkyl;
R⁴ is a linear or branched C₁ to C₅ alkyl;
R⁶ is a linear or branched C₁ to C₅ alkyl, which is unsubstituted or substituted by one or more substituents selected from OR^{8a}, ═O, and C₁ to C₃ alkyl;
R⁷ is a linear or branched C₁ to C₅ alkyl; and
R^{a} and R^{b} are selected from H and CH₃.

In particular embodiments of the invention, relating to monomers according to formula Ia or formula IIa:
R² is selected from H, CO₂R⁷. linear or branched C₂ to C₄ alkyl, linear or branched C₂ to C₃ alkenyl, where the latter two substituents are unsubstituted or substituted by one or more substituents selected from ═O, and C₁ to C₃ alkyl;
R³ is a linear or branched C₂ to C₅ alkyl;
R⁴ is C₁ alkyl;
R⁶ is a linear or branched C₂ to C₄ alkyl, which is unsubstituted or substituted by one or more substituents selected from C₁ to C₃ alkyl;
R⁷ is a linear or branched C₂ to C₄ alkyl; and
R^{a} and R^{b} are selected from H and CH₃.

In a fourth aspect of the invention, monomers according to the current invention may be selected from one or more in the following list:

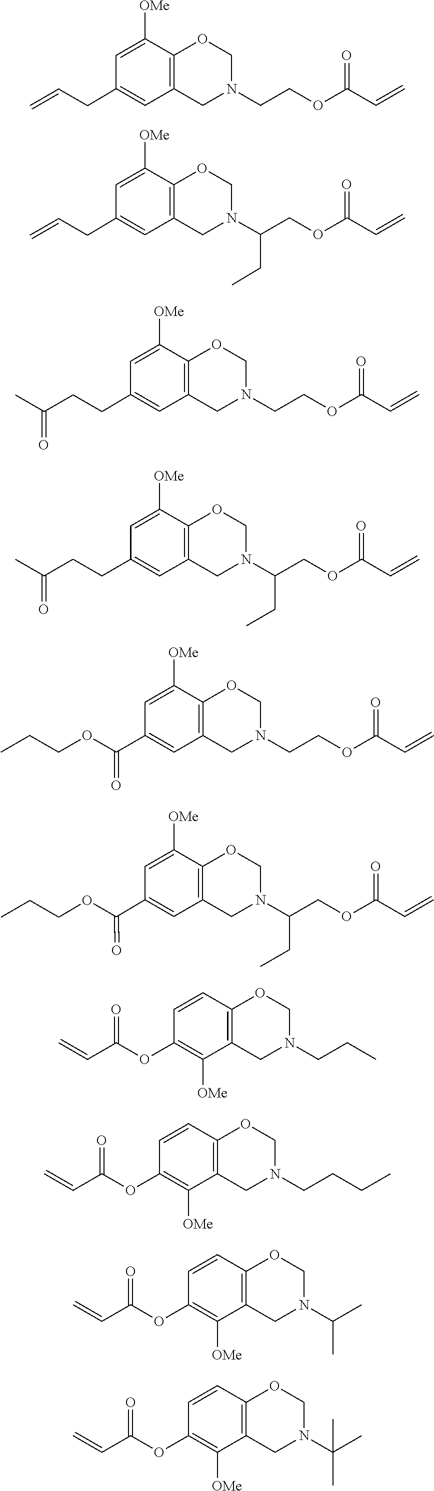

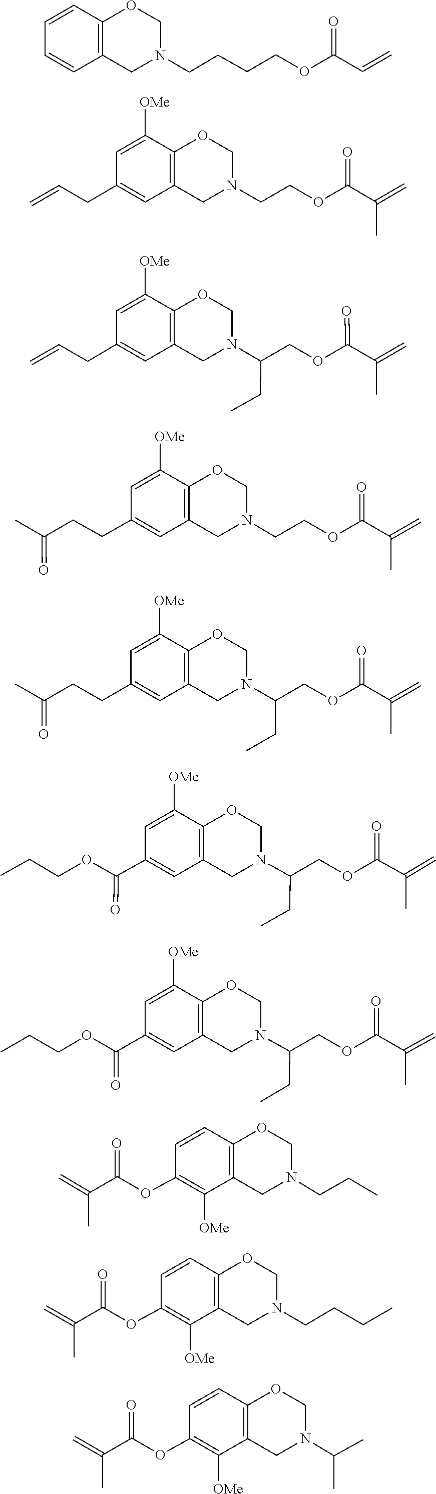

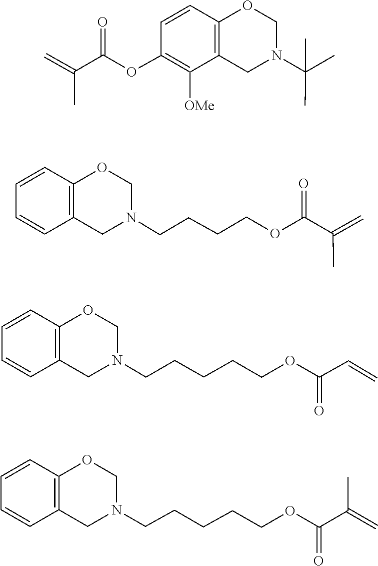 [SMILES: C=C(C)C(=O)Oc1ccc2c(c1OC)CN(C(C)(C)C)CO2 ; C=C(C)C(=O)OCCCCN1COc2ccccc2C1 ; C=CC(=O)OCCCCCN1COc2ccccc2C1 ; C=C(C)C(=O)OCCCCCN1COc2ccccc2C1]

As will be appreciated, the list above may also apply to the first to third aspects of the invention, where the monomers listed above may correspond to the monomers in the aspects.

As noted above, the polymeric products of the first and second aspects of the invention may be formed by a process that involves additive manufacturing. As such, there is also provided a formulation for additive manufacturing, comprising:
one or more monomers according to formula I or formula II:

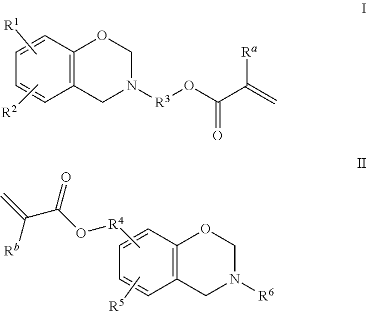

and a photoinitiator;
where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl.

As will be appreciated, the definitions set out in relation to formula I and formula II (and hence formula Ia and IIa) also apply to this aspect of the invention.

In particular embodiments of the invention, when the monomer is a compound of formula I where R³ is —CH₂CH₂—, then one of R¹ and R² may not be H.

In certain embodiments of the formulation, the formulation may further comprise one or more of an antioxidant, a stabiliser, a colourant, a diluent, a flame retardant, a plasticizer, a photoabsorber, a photoinhibitor, and a filler. As these materials are additives, one or more of these materials (e.g. a diluent and a filler) might not be required, for example, when the one or more monomers of formula I and/or II has a viscosity of less than 5 Pa/s, such as less than 2.5 Pa/s, such as less than 0.9 Pa/s, when measured as a neat monomer.

It will be appreciated that the formulation may also include further monomeric materials other than monomers of formula I and II (and Ia and IIa). However, such additional monomers should be compatible with the monomers and so should be an acrylate monomer of some kind. Suitable acrylate monomers that may be mentioned herein include, but are not limited to benzyl acrylate, butyl acrylate, ethyl acrylate, isobutyl acrylate, isobornyl acrylate, 2-ethylhexyl acrylate, methyl acrylate, tert-butyl acrylate, ethylene glycol diacrylate, 1,4-butanediol diacrylate, di(ethylene glycol) diacrylate, 1,6-hexanediol diacrylate, 1,3-butanediol dimethacrylate, ethylene glycol dimethacrylate, trimethylolpropane propoxylate triacrylate, pentaerythritol tetraacrylate, and combinations thereof.

As noted above, in the formulation, the monomers of formula I and formula II may be monomers according to the formula Ia and IIa as set out above. As these have already been discussed hereinbefore, they are not repeated here for the sake of brevity. This also applies to embodiments using the more particular list of substituents provided for R², R³, R⁴, R⁶, R⁷, R^{a}, and R^{b}. Particular monomers that may be mentioned herein may be selected from those in the list:

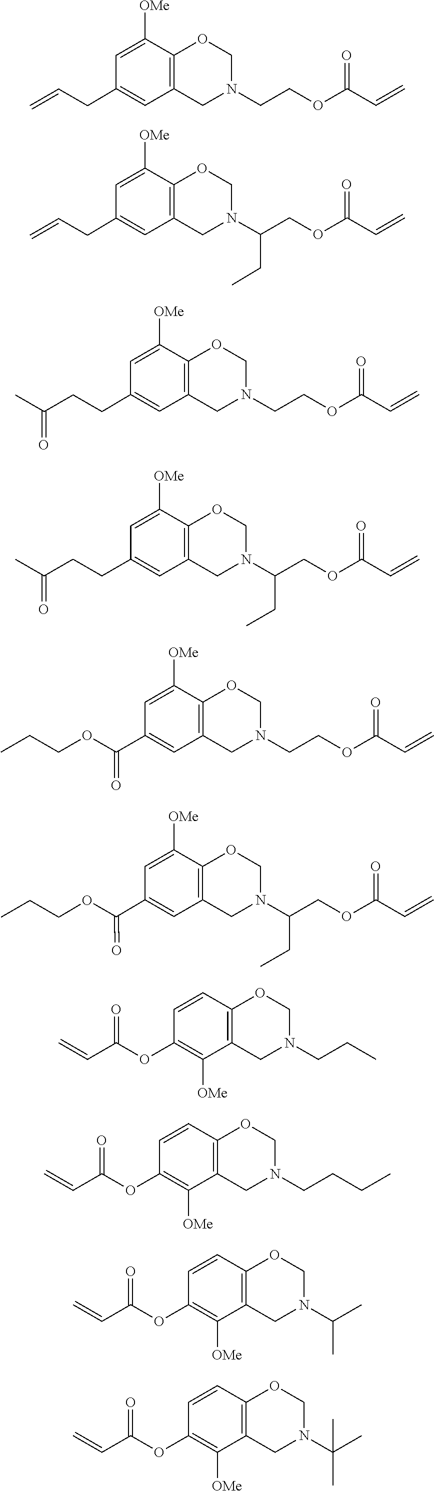

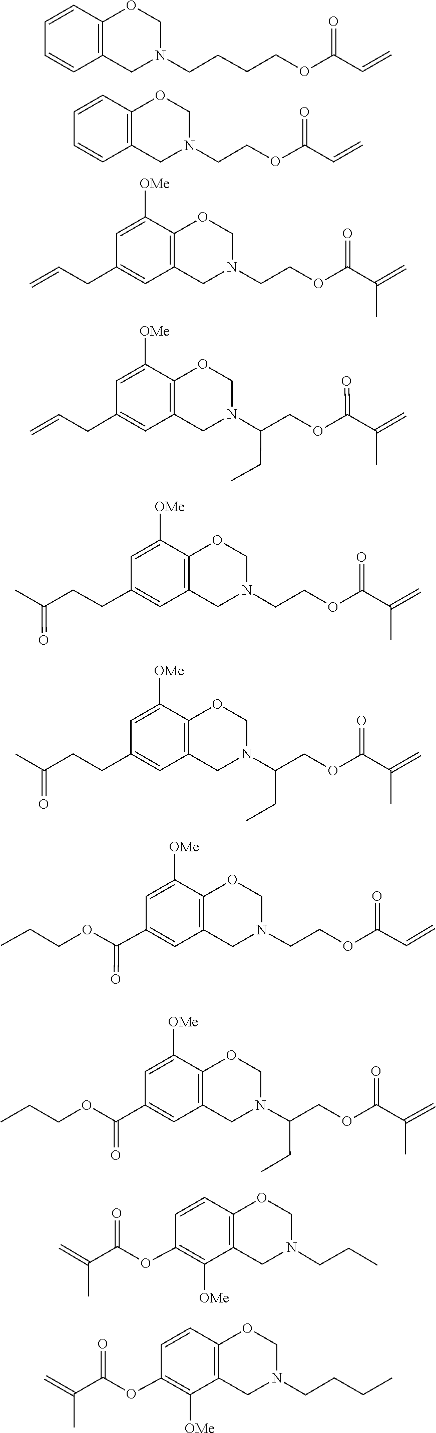

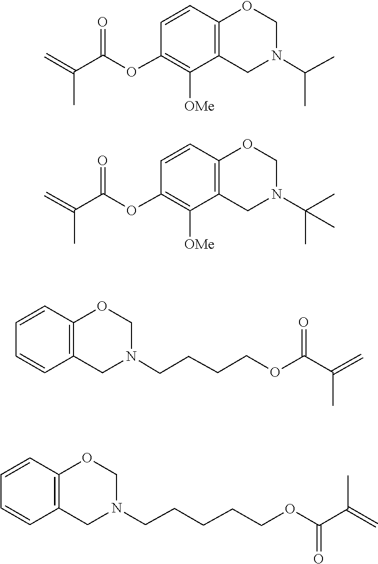

More particular monomers that may be mentioned in relation to the formulation may be selected from the list:

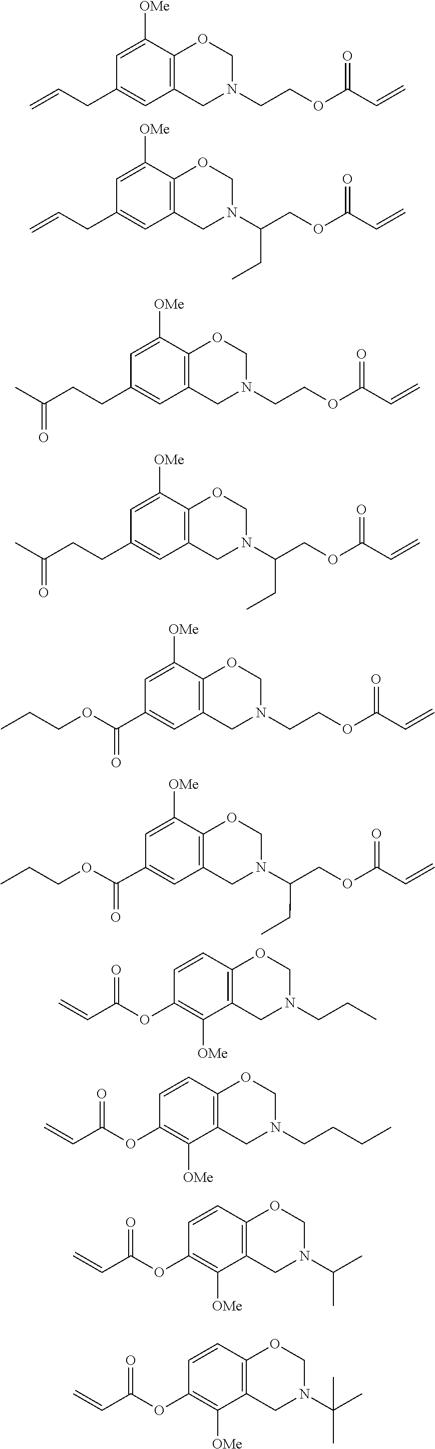

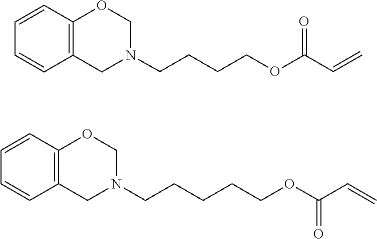 [SMILES: C=CC(=O)OCCCCN1COc2ccccc2C1 ; C=CC(=O)OCCCCCN1COc2ccccc2C1]

Yet particular monomers that may be mentioned in relation to the formulation may be selected from one or both (i.e. both) of:

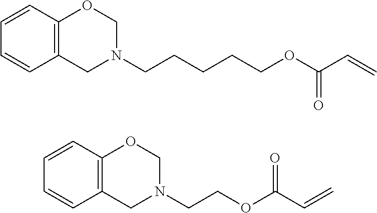 [SMILES: C=CC(=O)OCCCCCN1COc2ccccc2C1 ; C=CC(=O)OCCN1COc2ccccc2C1]

It will be appreciated that the lists monomers provided above may also apply to the other aspects of the invention.

As noted above, the monomers may be used in and the polymeric products produced from a method of additive manufacture. As such, there is provided a method of providing an intermediate product by additive manufacturing, the method comprising the steps of:
(a) 3D-printing an object one layer at a time according to a product design using a printing resin, where each layer is subjected to ultraviolet light for a first period of time before each further layer is added; and
(b) stopping the 3D-printing once the product design has been generated, thereby providing an intermediate product, wherein:
   the printing resin is either a monomer as described hereinbefore or a formulation as described hereinbefore; and the product is capable of being cured further using a thermal curing step.

As will be appreciated, in the method above, ultraviolet light is applied following the generation of each layer of the product in question. This may be for any suitable length of time as determined by the skilled person. It is noted that the selected ultraviolet wavelength may be selected by the skilled person using their knowledge of the field. Once the additive manufacturing process has been completed, the resulting intermediate product may be subjected to ultraviolet light for a second period of time to provide a further intermediate product. Again, any suitable second period of time and ultraviolet wavelength may be selected by the skilled person using their knowledge of the field. It is noted that the resulting intermediate products (whether subjected to the second burst of ultraviolet light or not) are not fully cured (i.e. still retain carbon-to-carbon double bonds that may be reacted further). Thus, either intermediate product may be subjected to a thermal curing step using a suitable temperature for a third period of time. Any suitable temperature and time may be used for this step and a person skilled in the filed may readily determine a suitable temperature and period of time. Following this thermal curing step it is believed that the product is fully cured, as defined hereinbefore.

In addition, there is provided a method of providing a final product by additive manufacturing, the method comprising the steps of:
(a) providing an intermediate product formed by an additive manufacturing process; and
(b) subjecting the intermediate product to a thermal curing step using a suitable temperature for a period of time, wherein
   the intermediate product is formed from polymeric matrix material, where the polymeric matrix material comprises repeating units derived from a monomer as described hereinbefore.

The intermediate product may be formed in the manner as described hereinbefore.

As will be appreciated, the final product obtained as described hereinbefore has high thermal stability and excellent mechanical properties. Therefore, the formulation and methods described hereinbefore allow efficient fabrication of high-performance thermosets for various demanding engineering applications.

Aspects and embodiments of the invention will now be discussed by reference to the following non-limiting examples.

### EXAMPLES

Materials

Phenol (99%), 2,2-bis(4-hydroxyphenyl)propane (BPA) (99%), paraformaldehyde (95%), 2-aminoethanol (99%), 5-amino-1-pentanol (95%) and phenylbis(2,4,6-trimethylbenzoyl)phosphine oxide (BAPO) were purchased from Sigma-Aldrich. A commercial resin (GR20) was provided by BMF Material Technology Inc. (Shen Zhen). All other chemicals were reagent grade and were purchased from Sigma-Aldrich, and used as received unless otherwise stated.

Analytical Techniques

Nuclear Magnetic Resonance (NMR) Spectroscopy

¹H spectra were performed on a JOEL ECA400 NMR spectrometer in deuterated CDCl₃ and tetramethyl silane was used as internal standard.

FT-IR Spectroscopy

FT-IR spectra were recorded by Perkin Elmer Frontier FTNIR/MIR spectrometers, with resolution of 4 cm⁻¹ for 16 scans.

Ultraviolet-Visible (UV-Vis) Spectrometry

The UV-vis absorption was conducted on a UV-vis spectrometer (Shimadzu Model: UV2700) in dilute chloroform solution.

Flow Viscosity

Flow viscosity experiments were carried out using a TA instruments Discovery Series Hybrid Rheometer DHR-3 with a parallel plate (diameter 25 mm) attachment at 25° C. with a shear rate ranging from 0.5 to 5 s⁻¹.

Field Emission Scanning Electron Microscopy (FESEM)

The surface morphology of fractured samples was studied by FESEM (JEOL JSM-7600F).

### Example 1

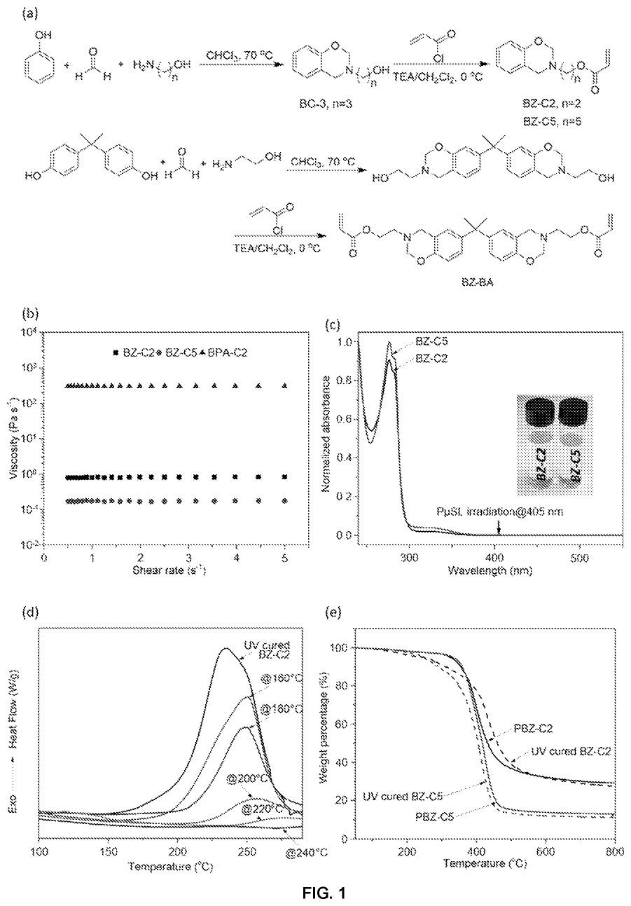

The synthetic approach to two monoacrylate-functionalized BZ-C2 and BZ-C5, and a diacrylate-functionalized BZ-BA is shown in FIG. 1a. Typical Mannich condensation was used to synthesize the hydroxyl-terminated BZs.

Paraformaldehyde (2.0 eq) and an amino alcohol (1.0 eq) selected from 2-aminoethanol and 5-amino-1-pentanol were added to a round bottom flask with stirring for 1 h. Then, chloroform was added, followed by the addition of phenol (1.0 eq) or BPA. The reaction mixture was heated to 70° C. and reacted overnight. After cooling to room temperature, an extraction process was conducted with a sodium hydroxide solution (0.1 N) to remove unreacted acidic impurities. The extracted organic layer was dried over sodium sulphate, filtered and removed under vacuum to yield the desired hydroxyl-BZ precursors.

BZ-C2 Precursor

BZ-C2 precursor was prepared from 2-aminoethanol and phenol by following the protocol above.

¹H NMR (400 MHz, CDCl₃): δ (ppm) 7.13-6.77 (m, 4H, aromatics), 6.42 (dd, 1H), 6.14 (q, 1H), 5.85 (dd, 1H), 4.85 (s, 2H), 4.32 (t, 2H), 4.05 (s, 2H), 3.07 (t, 2H).

BZ-C5 Precursor

BZ-C5 precursor was prepared from 5-amino-1-pentanol and phenol by following the protocol above.

¹H NMR (400 MHz, CDCl₃): δ (ppm) 7.09-6.75 (m, 4H, aromatics), 6.38 (dd, 1H), 6.10 (q, 1H), 5.79 (dd, 1H), 4.83 (s, 2H), 4.14 (t, 2H), 3.96 (s, 4H), 2.73 (t, 2H), 1.71-1.41 (m, 6H).

BZ-BA Precursor

BZ-BA precursor was prepared from 2-aminoethanol and BPA by following the protocol above except the molar ratio of paraformaldehyde, 2-aminoethanol and BPA was 4:2:1.

¹H NMR (400 MHz, CDCl₃): δ (ppm) 7.27-6.93 (m, 6H, aromatics), 6.56 (dd, 2H), 6.25 (q, 2H), 6.02 (dd, 2H), 4.88 (s, 4H), 4.37 (t, 4H), 4.10 (s, 4H), 3.11 (t, 4H), 1.57 (s, 6H).

BC-3

BC-3 was prepared from 3-aminoethanol and phenol by following the protocol above.

¹H NMR (400 MHz, CDCl₃): δ (ppm) 7.1-6.77 (m, 4H, aromatics), 4.87 (s, 2H), 3.99 (s, 2H), 2.72 (t, 2H), 1.60 (m, 2H), 0.94 (t, 3H).

### Example 2

To a solution of the acrylic acid (1.1 eq.) in dry CH₂Cl₂ at 0° C., oxalyl chloride ((COCl)₂, 1.1 eq.) was added dropwise, followed by the addition of a catalytic amount of dry DMF (2 drops). Then, the solution was allowed to stir at room temperature for 3 h. The solvent was removed under reduced pressure to afford crude acryloyl chloride that was directly used in the next step.

To an ice-bath cooled CH₂Cl₂ solution with hydroxyl-BZ (prepared in Example 1, 1 eq.) and dried triethyl amine (TEA, 1.1 eq.), the crude acryloyl chloride was added to the reaction mixture slowly. The solution was brought to room temperature and continuously stirred for another 4 h. Then, the CH₂Cl₂ solution was washed with saturated NaHCO₃ solution thrice and deionised (DI) water once. The CH₂Cl₂ layer was collected, dried, filtered and the CH₂Cl₂ solvent was removed under vacuum to obtain the desired BZ monomers.

BZ-C2 Monomer

BZ-C2 monomer was prepared from BZ-C2 precursor by following the protocol above (yellow liquid, yield: 82%).

¹H NMR (400 MHz, CDCl₃): δ (ppm) 7.13-6.77 (m, 4H, aromatics), 6.42 (dd, 1H), 6.14 (q, 1H), 5.85 (dd, 1H), 4.85 (s, 2H), 4.32 (t, 2H), 4.05 (s, 2H), 3.07 (t, 2H). FTIR (KBr, cm⁻¹): 1720 (C═O st), 1638 (C═C st), 1490 (C—C Ar st), 1224 (C—O—C st asymmetric), 1063 (C—O—C st symmetric), 933 (N—C—O st).

BZ-C5 Monomer

BZ-C5 monomer was prepared from BZ-C5 precursor by following the protocol above (yellow 30 liquid, yield: 86%).

¹H NMR (400 MHz, CDCl₃): δ (ppm) 7.09-6.75 (m, 4H, aromatics), 6.38 (dd, 1H), 6.10 (q, 1H), 5.79 (dd, 1H), 4.83 (s, 2H), 4.14 (t, 2H), 3.96 (s, 4H), 2.73 (t, 2H), 1.71-1.41 (m, 6H). FTIR (KBr, cm⁻¹): 1723 (C═O st), 1635 (C═C st), 1487 (C—C Ar st), 1226 (C—O—C st asymmetric), 1059 (C—O—C st symmetric), 928 (N—C—O st).

BZ-BA Monomer

BZ-BA monomer was prepared from BZ-BA precursor by following the protocol above except the molar ratio of acrylic acid, TEA and BZ-BA precursor was 2.2:2.2:1 (highly viscous orange liquid, yield: 78%).

¹H NMR (400 MHz, CDCl₃): δ (ppm) 7.27-6.93 (m, 6H, aromatics), 6.56 (dd, 2H), 6.25 (q, 2H), 6.02 (dd, 2H), 4.88 (s, 4H), 4.37 (t, 4H), 4.10 (s, 4H), 3.11 (t, 4H), 1.57 (s, 6H).

Results and Discussion

The characteristic proton resonances (Ar—CH₂—N— and —O—CH₂—N—) of oxazine ring appear at 4.85 and 4.83 ppm for BZ-C2, and 4.05 and 3.96 ppm for BZ-C5. The multiplets in the range of 7.13-6.77 ppm and 7.09-6.75 ppm are assigned to their aromatic protons. The vinyl protons of BZ-C2 are observed at 6.42, 6.14 and 5.85 ppm. The vinyl protons of BZ-C2 are observed at 6.38, 6.10 and 5.79 ppm. The structure of BZ-BA is verified with ¹H NMR as well.

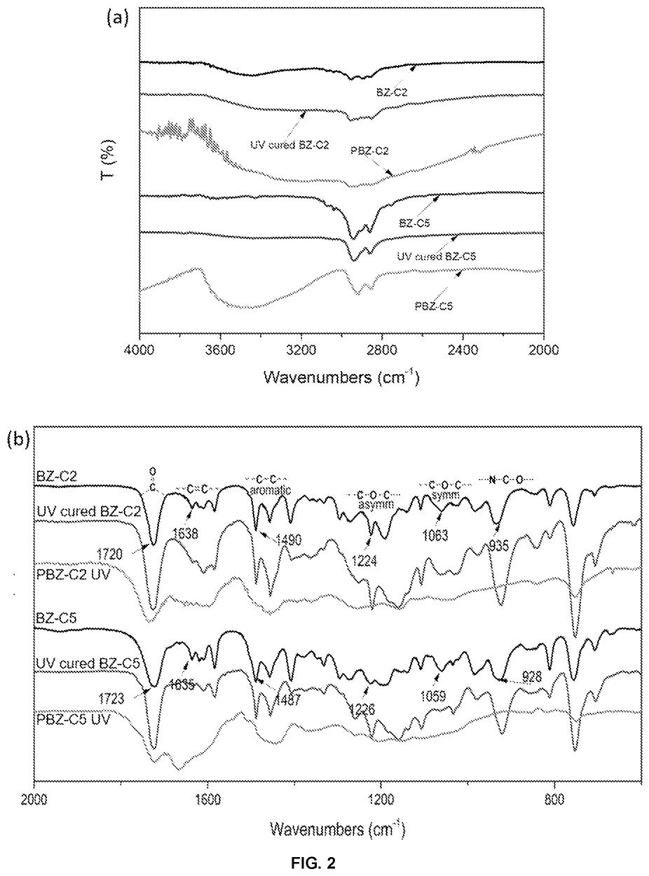

FT-IR absorption further confirms the chemical structures of BZ-C2 and BZ-C5. As seen from FIG. 2a, the characteristic absorption band of C—O—C and N—C—O of oxazine ring are clearly observed at ˜1224 and ˜935 cm⁻¹. Meanwhile, the stretching absorption of carbonyl and vinyl groups in acrylate structure are found at around 1718 and 1638 cm⁻¹. These results prove the successful synthesis of the acrylate BZs.

### Example 3

The viscosity and UV-vis absorption data of BZ monomers (prepared in Example 2) were collected.

Results and Discussion

FIG. 1b shows that the viscosity of BZ-BA, BZ-C2 and BZ-C5 is 300, 0.9 and 0.1 Pa·s⁻¹, respectively. Unsurprisingly, the diacrylate BZ-BA monomer has a much higher viscosity than the monoacrylate-based BZ-C2 and BZ-C5 due to its comparatively large molecule structure. It is reported that the viscosity of a photopolymerization printable formulation should be on the order of 5 Pa·s⁻¹. Otherwise, the printing resin is not able to flow sufficiently and recover the building platform evenly if the viscosity is higher than this practical value (Melchels, F. P. W., Feijen, J. & Grijpma, D. W. Biomaterials 2010, 31, 6121-6130). Therefore, due to its highly viscous nature, BZ-BA is not suitable for the formulation of a photo-curable resin without adequate addition of monomer diluents. Moreover, BZ-BA had poor stability in air and gelatinize within several hours.

In contrast, the monoacrylate BZ-C2 and BZ-C5 were fairly stable and possessed intrinsic low viscosity, which is favourable for photo-curable resins and for improving the printing facility and resolution. Compared to BZ-C2, BZ-C5 was found to have a more significantly low viscosity that is about 10 times lower than BZ-C2. In FIG. 1c, the UV-vis absorption of BZ-C2 and BZ-C5 in dilute CH₃Cl are shown and both of them exhibited similar absorption bands with a maximum absorption at 277 nm. Since BZ-C2 and BZ-C5 have no UV-vis absorption beyond 400 nm, the resins based on them will not affect the light penetration of the UV light source with a wavelength longer than 400 nm.

### Example 4

Resin Formulation Based on BZ-C2 Monomer

BZ-C2 monomer (prepared in Example 2), BAPO (0.45 wt %) and THF (30 wt %) were mixed together and homogenized with a vortex mixer for 30 s. The resultant mixture was subsequently allowed to stand at room temperature for 2 h to ensure the absence of bubbles.

Resin Formulation Based on BZ-C5 Monomer

BAPO (0.6 wt %) was dissolved in a miniscule amount of THF and added to BZ-C5 monomer (prepared in Example 2). The mixture was mixed homogeneously using a vortex mixer for 30 s and was allowed to stand for 2 h to ensure the absence of bubbles.

### Example 5

To obtain PBZ structures of the two BZ monomers, thermal polymerization of BZ-C2 and BZ-C5 monomers was carried out and examined using DSC analysis.

PBZ-C2 and PBZ-C5

PBZ-C2 and PBZ-C5 samples for FT-IR, DSC, TGA and DMA analyses, and 3-point bending test were prepared by first photocuring the uniform samples with UV, followed by thermal curing with a progressive heat treatment. Typically, the liquid BZ-C2 or BZ-C5 resin formulation (prepared in Example 4) was added into silicone moulds with a rectangular cavity, and photocured within a UV chamber (2 mW cm⁻²) for 3 min. The specimen bars were demoulded, flipped over and photocured within the UV chamber for another 3 min. The specimen thickness was controlled by the resin adding volume. The thermal curing was subsequently carried out by subjecting the photocured samples to the following heating schedule: 140° C. (1 h), 160° C. (1 h), 180° C. (1 h), 200° C. (1 h), 220° C. (1 h) and 240° C. (1 h), to give PBZ-C2 or PBZ-C5.

DSC Analysis

DSC (TA Instruments 2010) was performed from room temperature to 300° C. at a constant heating rate of 10° C./min under N₂ atmosphere.

Results and Discussion

The resin was deposited in a silicone mould and it solidified within ˜10 s under UV irradiation. The photo curing process was continued until full cure of the samples occurred, as shown by the significantly decreased FTIR absorption of the vinyl structure (FIG. 2). Afterwards, progressive thermal treatment was applied to the photocured sample and a specimen was collected at each curing stage for DSC scanning. As presented in FIG. 1d, the photocured BZ-C2 exhibited a broad exothermic peak at 235° C. (onset at 162° C.), corresponding to the oxazine ring-opening polymerization. Being thermally treated from 160 to 220° C., the exothermic peak decreased gradually at each stage, indicating the consecutive ring-opening of BZs and the incomplete PBZ network within the sample. When the curing temperature reached 240° C., the exotherm disappeared completely, suggesting that an entire conversion to the PBZ network has occurred. Therefore, the maximum treatment temperature of 240° C. was applied to all thermal curing processes in the following examples.

### Example 6

The thermal stability of the BZ-C2, BZ-C5, PBZ-C2 and PBZ-C5 was examined using TGA analysis in a N₂ atmosphere.

TGA Measurements

TGA measurements were performed on a TA Instruments 2950 under N₂ atmosphere at a heating rate of 10° C./min.

Results and Discussion

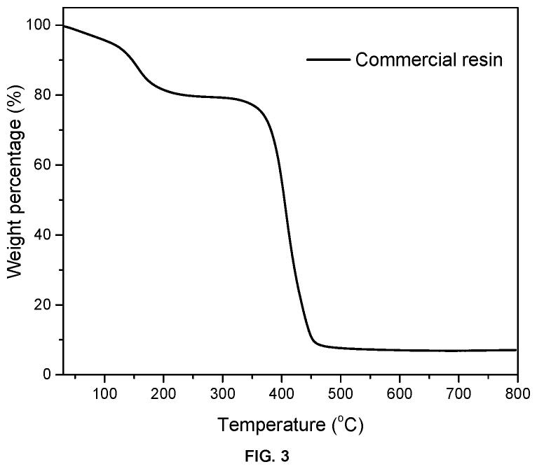

FIG. 1e shows that the 5 and 10% weight loss temperatures (T₅ and T₁₀) of photocured BZ-C2 are 252 and 314° C., respectively, which are significantly improved to 326 and 358° C., respectively, for the resultant PBZ-C2. Similar findings were observed for photocured BZ-C5 as well, with the T₅ and T₁₀ enhanced from 249 and 292° C. to 327 and 360° C., respectively, after thermal curing. The greatly improved thermal stability of PBZs is attributed to the highly crosslinked network in their structures. Interestingly, both PBZ-C2 and PBZ-C5 exhibited similar initial degradation temperature because the network degradation is always initiated from the cleavage of their Mannich base formed by ring-opening. After the initial degradation, all the tested samples showed a second thermal degradation attributed to the thermal decomposition of polymer main chains. It is worth noting that the char yield at 800° C. for PBZ-C2 is ˜30%, which is much higher than that of the commercial resin (˜7%) (FIG. 3), suggesting the potential applications of flame retardants and carbon precursors for PBZ-C2.

### Example 7

The thermomechanical behaviours of photocured BZ-C2/C5 and PBZ-C2/C5 in Example 5 were studied using dynamic mechanical analysis (DMA) and 3-point bending test.

DMA

DMA was carried out with a TA instruments Q800 DMA utilizing the single cantilever mode with temperature ramp from room temperature to 280° C.

3-Point Bending Test

The flexural properties were measured by three-point bending tests using a mechanical tester Instron 5567 with loading speed 1 mm/min.

Results and Discussion

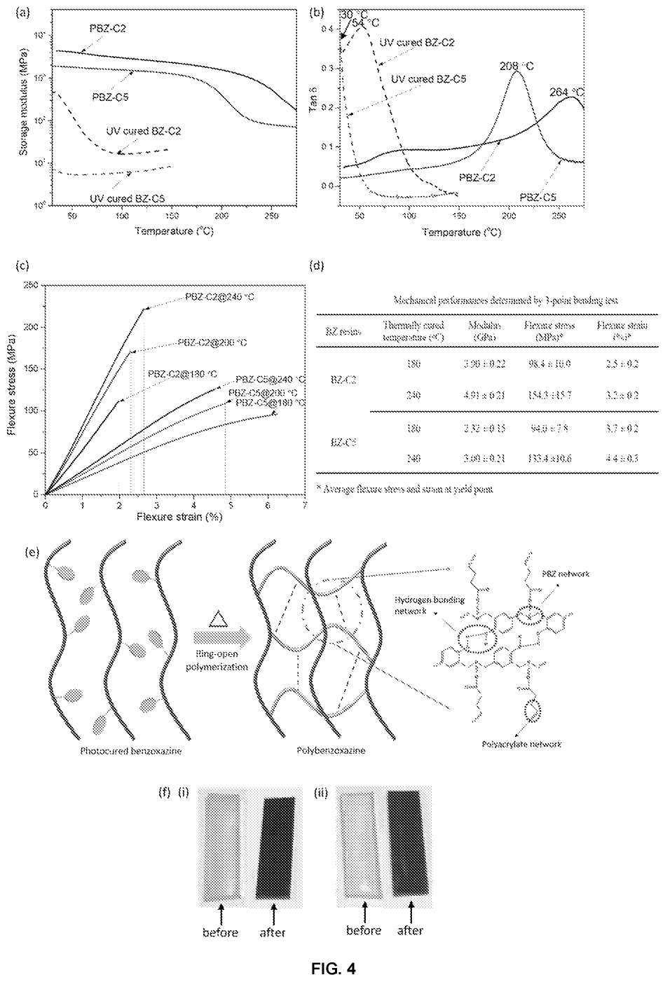

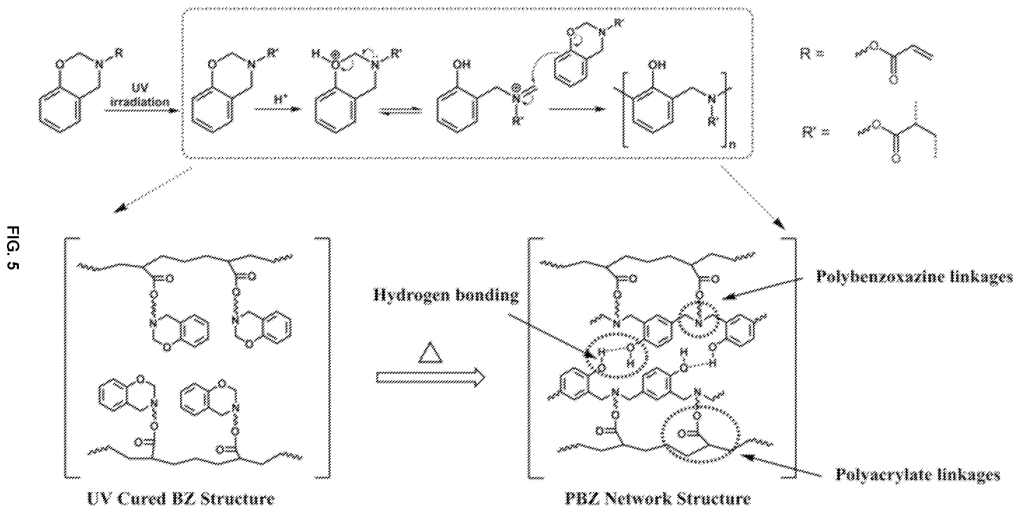

FIG. 4a-b show the temperature dependence of storage modulus and tan δ, where the maximum value in the tan δ curve is used to determine T_{g}. For the photocured BZ-C2 and BZ-C5, the initial storage modulus was 478 and 7 MPa, respectively, and these values decreased sharply as the temperature increased, indicating the elastomer behaviour of the samples. On the other hand, PBZ-C2 and PBZ-C5 exhibited remarkably enhanced initial storage modulus of 4.3 and 1.9 GPa which were maintained up to nearly 150° C. Moreover, a T_{g} of 54° C. and less than 30° C. was found for photocured BZ-C2 and BZ-C5, respectively, whereas PBZ-C2 and PBZ-C5 showed highly improved T_{g} of 264 and 208° C., respectively. Considering the dual curing process of photo and thermal photopolymerization as well as the intermolecular hydrogen bonding formed (FIG. 5), an extremely crosslinked triple network (FIG. 4e) is established eventually within the PBZ structures, and this contributes mostly to the significantly enhanced storage modulus and T_{g}. In particular, the broad tan δ peaks of PBZ-C2 and PBZ-C5 curves suggest that there is a heterogenous network in their structures that consists of both highly and loosely crosslinked regions.

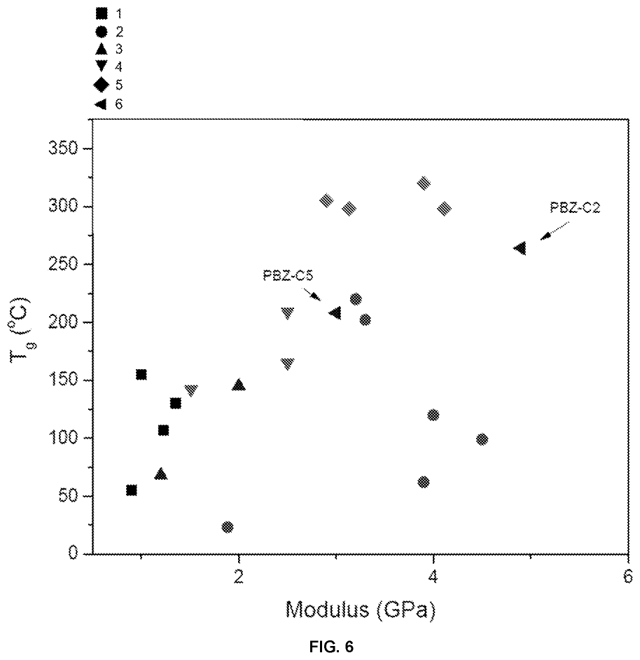

Encouraged by the DMA results, the mechanical performances of PBZ-C2 and PBZ-C5 were further studied with 3-point bending test. Their representative flexure stress-strain curves are depicted in FIG. 4c-d. As shown, the flexure modulus of PBZs was greatly improved with progressive thermal treatment due to increased crosslinking of the samples. Remarkable flexure modulus of 4.91 GPa and 3.02 GPa were achieved for the fully cured PBZ-C2 and PBZ-C5, respectively. PBZ-C2 exhibited a T_{g} as high as 264° C. and a remarkable flexural modulus of 4.91 GPa due to the highly cross-linked triple network within its structure. Compared to commonly used plastic resins, both PBZ-C2 and PBZ-C5 demonstrate highly enhanced T_{g} and moduli which are associated with photo printability (FIG. 6, see below for the detailed description of the legend). These results show that both PBZ-C2 and PBZ-C5 are high-performance thermosets with excellent mechanical properties.

Detailed Description of the Legend in FIG. 6
▪: 1—Acrylate resins (B. Zhang et al., Nat. Commun. 2018, 9, 1831; S. Deng et al., Adv. Mater. 2019, 31, 1903970; and R. Ding et al., Polym. Chem. 2019, 10, 1067-1077)
•: 2—Epoxy resins (A. Trogé et al., J. Acoust. Soc. Am. 2010, 128, 2704-2714; Q. Nguyen et al., J. Appl. Polym. Sci. 2020, 137, e49051; and R. Feng & R. J. Farris, J. Mater. Sci. 2002, 37, 4793-4799)
▴: 3—Hybrid resins (X. Kuang et al., Sci. Adv. 2019, 5, eaav5790; and X. Kuang et al., Macromol. Rapid Commun. 2018, 39, 1700809)
▾: 4—Phenolic resins (P. Jahanmard & A. Shojaei, RSC Adv. 2015, 5, 80875-80883; J. Zhou et al., Poly. Compos. 2013, 34, 1245-1249; and F. Cardona, A. L. Kin-Tak & J. Fedrigo, J. Appl. Poly. Sci. 2012, 123, 2131-2139)
♦: 5—Cyanate ester resins (S. Gangulia et al., Polymer 2003, 44, 1315-1319; I. Mondragón et al., Polymer 2006, 47, 3401-3409; X. Gu et al., Chem. Eng. J. 2016, 298, 214-224; and H. Li et al., High Perform. Polym. 2018, 30, 38-46)
[symbol] : 6—This work

### Example 8

Due to the low viscosity and interesting features of the resultant PBZs, BZ-C2 and BZ-C5 monomers were formulated into photo resins to demonstrate their use in manufacturing high-performance PBZ thermosets with a two-stage fabrication process, consisting of PμSL 3D printing and post thermal curing.

PμSL Printing with BZ-C2 or BZ-C5 Based Resin Formulation Prepared in Example 4

The PμSL printing process was performed with a commercially available 3D printer (nanoArch S140, BMF). A UV-LED (405 nm) was utilized as the light source. An intensity of 17.5 mW cm⁻² was used in all the printing processes. Computer aided design (CAD) of the print structures were designed in the software of Autodesk fusion 360. The resulting STL files were sliced for a 2D file output using BMF PμSL printing software with different slicing thickness. After printing, the acquired objects were washed thoroughly with isopropanol to remove any residual unreacted resin. After that, they were left to dry for 5 min and then placed into a UV curing chamber for further photopolymerization for 5 min. The printed 3D structures were placed into a vacuum oven at 60° C. overnight to remove residual solvent.

Preparation of PBZ 3D Structured Objects

The fully dried 3D structured objects prepared above were taken for thermal treatment as described in Example 5 to achieve the final PBZ products. The heights of the 3D structured objects were measured thrice using a micrometre calliper, before and after the thermal treatment.

Results and Discussion

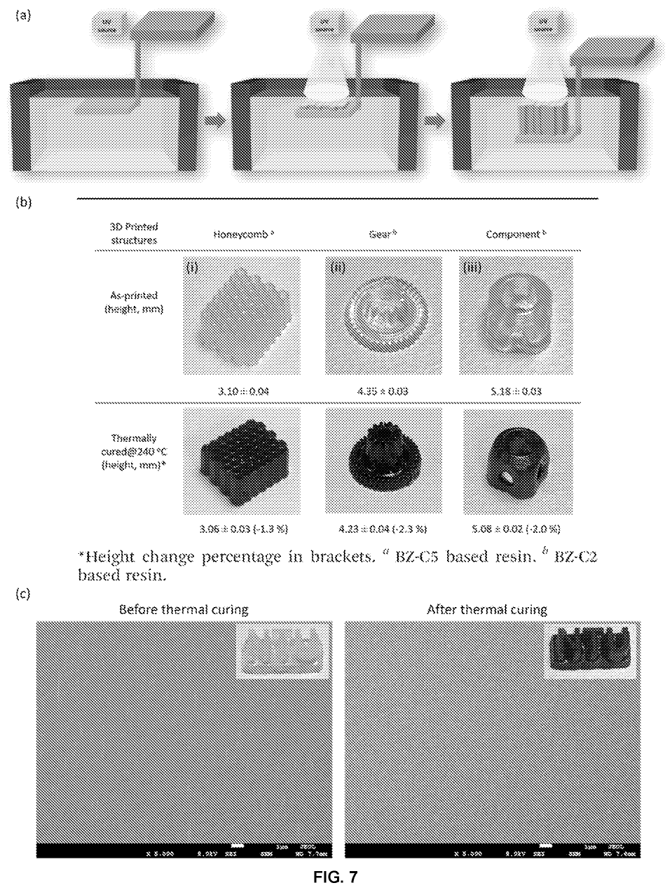

FIG. 7a depicts a schematic of PμSL printing of BZ-C2 or BZ-C5 photo resins with a UV light source (405 nm, 17.5 mW/cm²). The formulated resin was exposed to UV irradiation for 6 s per layer and generated a designed pattern with roughly 100 μm thicknesses. As the printing process progressed, the structural geometry was built up in a layer-by-layer manner. Once the printing was completed, the structure was removed, photocured further and transferred to a second stage thermal curing. FIG. 7b demonstrates the printed objects with various 3D structures before and after thermal treatment. Measured using a micrometre calliper, only small height shrinkages were detected for the resultant PBZ structures. As seen from the SEM scans in FIG. 7c, there were no bubbles or voids on the PBZ fracture surfaces, suggesting a smooth post cure without release of volatile compounds. The results show that the formulation based on BZ-C2 and BZ-C5 monomers is suitable for PμSL 3D printing, and consequently, high-performance PBZ structured objects can be achieved. Further, this work expands the existing library with a new class of PBZs for popular additive manufacturing.

### Example 9

BC-3 was synthesized in Example 1 and used as a diluent for PμSL printing with BZ-C2 or BZ-C5 based resin formulation described in Example 4. The photocurable behaviour and printability were evaluated.

BZ-C2:BC3(20%/30%) Resin Formulation

BAPO (1 wt %) was dissolved in a miniscule amount of THF and added to the mixture of BZ-C2 monomer and BC3 monomer (20 wt % or 30 wt % of BZ-C2). The mixture was mixed homogeneously using a vortex mixer for 30 s and was allowed to stand for 2 h to ensure the absence of bubbles.

BZ-C5:BC3(20%/30%) Resin Formulation

BZ-C5:BC3(20%/30%) resin formulation was prepared from BZ-C5 monomer and BC3 monomer by following the protocol for BZ-C2:BC3 (20/30%) resin formulation.

Results and Discussion

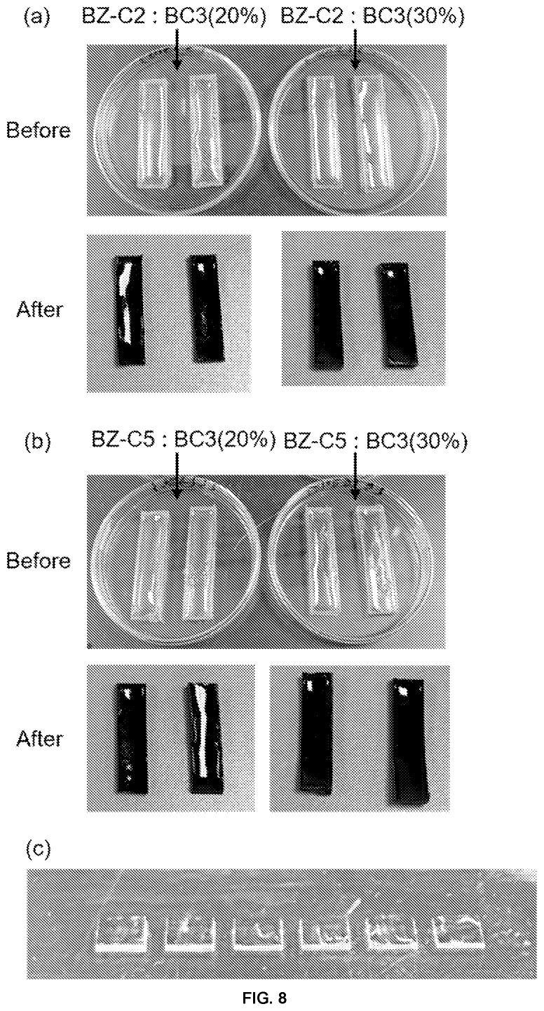

FIG. 8a-b show that BZ-C2 or BZ-C5 based resin formulation are curable and printable with BC3 diluent. FIG. 8c shows the printed patterns of BZ-C2 with 30 wt % BC3.

### Example 10

3D printable BZs can blend with other acrylates to form new photo resins. Therefore, a blend of BZ-C5 and 1,6-hexanediol diacrylate (HDODA) was prepared and taken for TGA analysis as described in Example 6.

BZ-C5:HDODA Blend

BAPO (0.4 wt %) was dissolved in a miniscule amount of THF and added to the mixture of BZ-C5 monomer and HDODA monomer (1:1, w:w). The mixture was mixed homogeneously using a vortex mixer for 30 s and was allowed to stand for 2 h to ensure the absence of bubbles.

Results and Discussion

TABLE 1
TGA results of neat BZ-C5 and BZ-C5:HDODA (1:1
wt) affording 5% weight loss from samples before
and after thermal post curing after UV curing.

| | Material | | | |
|---|---|---|---|---|
| | Neat BZ-C5 | | BZ-C5 and BZ-C5:HDODA (1:1 wt) | |
| | Processing methods | | | |
| | | Thermally | | Thermally |
| | UV | Cured @ | UV | Cured @ |
| | Cured | 240° C. | Cured | 240° C. |
| Temp (° C.) | 275 | 321 | 336 | 332 |
| with 5% | | | | |
| weight loss | | | | |

### Example 11

Various 3D printable BZs can blend with each other to form new photo resins. Therefore, a blend of BZ-C2 and BZ-C5 was prepared and taken for viscosity, TGA analysis, and mechanical studies as described in Examples 6 and 7.

BZ-C2 and BZ-C5 Blend

Resin formulation preparation: BAPO (1 wt %) is dissolved in a miniscule amount of THF and is added to a mixture of BZ-C2 monomer and BZ-C5 monomer in various weight ratios (e.g., 1:3, 1:1, 3:1). The mixture is mixed homogeneously using a vortex mixer for 30 seconds and is allowed to stand for 2 h to ensure the absence of bubbles.

Results and Discussion

TABLE 3
Properties of BZ-C2 and BZ-C5 blend.

| | Determined properties¹ | | | | |
|---|---|---|---|---|---|
| | Neat | Neat | Projected properties² | | |
| | BZ-C2 | BZ-C5 | Ratio of BZ-C2:BZ-C5 (w:w) | | |
| | (100%) | (100%) | 25:75 | 50:50 | 75:25 |
| Viscosity | 0.9 | 0.1 | 0.3 | 0.5 | 0.7 |
| (Pa · s⁻¹) | | | | | |
| T_{d} (° C.) | 326 | 327 | 326 | 326 | 326 |
| Flexure strength | 154.3 | 133.4 | 138.6 | 143.9 | 149.1 |
| (MPa) | | | | | |
| Flexure modulus | 4.91 | 3.0 | 3.48 | 3.96 | 4.43 |
| (GPa) | | | | | |

¹from this work;
²estimated from values of this work.

It is believed that a blend containing BZ-C2 and BZ-C5 would be useful and the expected properties for such blends are presented in Table 3 above. It is expected that blends of the two materials will allow one to obtain the desired physical properties for any desired application—particularly the viscosity of the blended material.

1. A polymeric product, wherein the polymeric product is formed from a cured polymeric material that comprises a repeating unit derived from a monomer according to formula I or formula II: 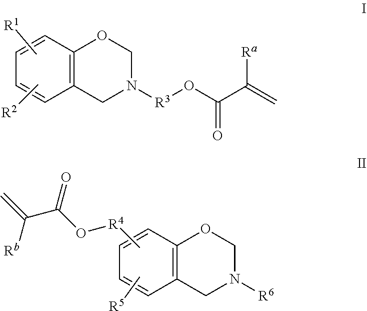
where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
wherein the polymeric product has been subjected to photocuring followed by thermal curing.

2. A polymeric product, wherein the polymeric product is formed from a partly-cured polymeric material that comprises a repeating unit derived from a monomer according to formula I or formula II: 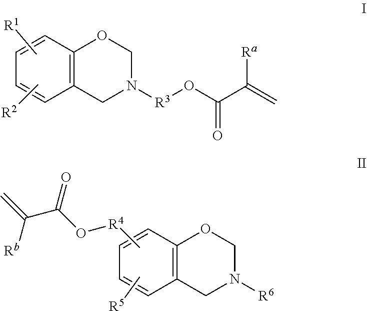
where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
wherein the polymeric product has been subjected to photocuring.

3. The polymeric product according to claim 1, wherein the monomer has a viscosity of less than 5 Pa/s when measured as a neat monomer.

4. A monomer according to formula I or formula II: 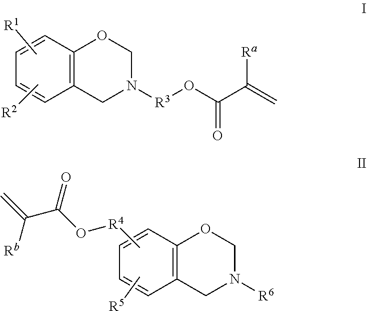
where:
each R¹, R², R⁵, R⁶, R^{a} and R^{b} is a substituent independently selected from H, CO₂R⁷. linear or branched C₁ to C₂₀ alkyl, C₃ to C₁₄ cycloalkyl, linear or branched C₂ to C₂₀ alkenyl, C₃ to C₁₄ cycloalkenyl, linear or branched C₂ to C₂₀ alkynyl, C₅ to C₁₄ cycloalkynyl, aryl, and heteroaryl, where each of these latter eight substituents is unsubstituted or substituted by one or more substituents selected from halo, OR^{8a}, CO₂R^{8b}, ═O, COR^{8c}, NR^{8d}R^{8e}, and C₁ to C₃ alkyl;
each R³ and R⁴ is independently a linear or branched C₁ to C₂₀ alkyl group, which group is unsubstituted or substituted by one or more substituents selected from halo, OR^{9a}, CO₂R^{9b}, ═O, COR^{9c}, NR^{9d}R^{9e}, C₁ to C₃ alkyl, aryl, and heteroaryl,
R⁷ is a linear or branched C₁ to C₂₀ alkyl group, which group is unsubstituted or substituted by one or more substituents selected from halo, OR^{10a}, CO₂R^{10b}, ═O, COR^{10c}, NR^{10d}R^{10e}, C₁ to C₃ alkyl, aryl, and heteroaryl,
each R^{8a} to R^{8e}, R^{9a} to R^{9e} and R^{10a} to R^{10e} is independently selected from H or a linear or branched C₁ to C₅ alkyl,
provided that when the monomer is a compound of formula I where R³ is —CH₂CH₂—, then one of R¹ and R² is not H.

5. The monomer according to claim 4, wherein the monomer has a viscosity of less than 5 Pa/s when measured as a neat monomer.

6. The monomer according to claim 4, wherein the monomer according to formula I or formula II is a monomer according to formula Ia or formula IIa: 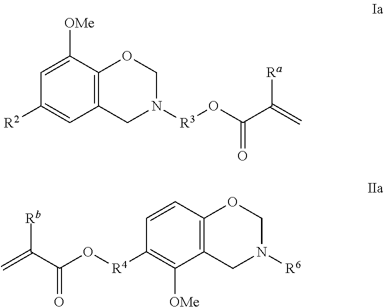
where:
R² is selected from H, CO₂R⁷. linear or branched C₁ to C₅ alkyl, linear or branched C₂ to C₅ alkenyl, where the latter two substituents are unsubstituted or substituted by one or more substituents selected from OR^{8a}, ═O, and C₁ to C₃ alkyl;
R³ is a linear or branched C₂ to C₅ alkyl;
R⁴ is a linear or branched C₁ to C₅ alkyl;
R⁶ is a linear or branched C₁ to C₅ alkyl, which is unsubstituted or substituted by one or more substituents selected from OR^{8a}, ═O, and C₁ to C₃ alkyl;
R⁷ is a linear or branched C₁ to C₅ alkyl; and
R^{a} and R^{b} are selected from H and CH₃.

7. The monomer according to claim 6, wherein:
R² is selected from H, CO₂R⁷. linear or branched C₂ to C₄ alkyl, linear or branched C₂ to C₃ alkenyl, where the latter two substituents are unsubstituted or substituted by one or more substituents selected from ═O, and C₁ to C₃ alkyl;
R³ is a linear or branched C₂ to C₅ alkyl;
R⁴ is C₁ alkyl;
R⁶ is a linear or branched C₂ to C₄ alkyl, which is unsubstituted or substituted by one or more substituents selected from C₁ to C₃ alkyl;
R⁷ is a linear or branched C₂ to C₄ alkyl; and
R^{a} and R^{b} are selected from H and CH₃.

8. A monomer according to claim 4 selected from the list:

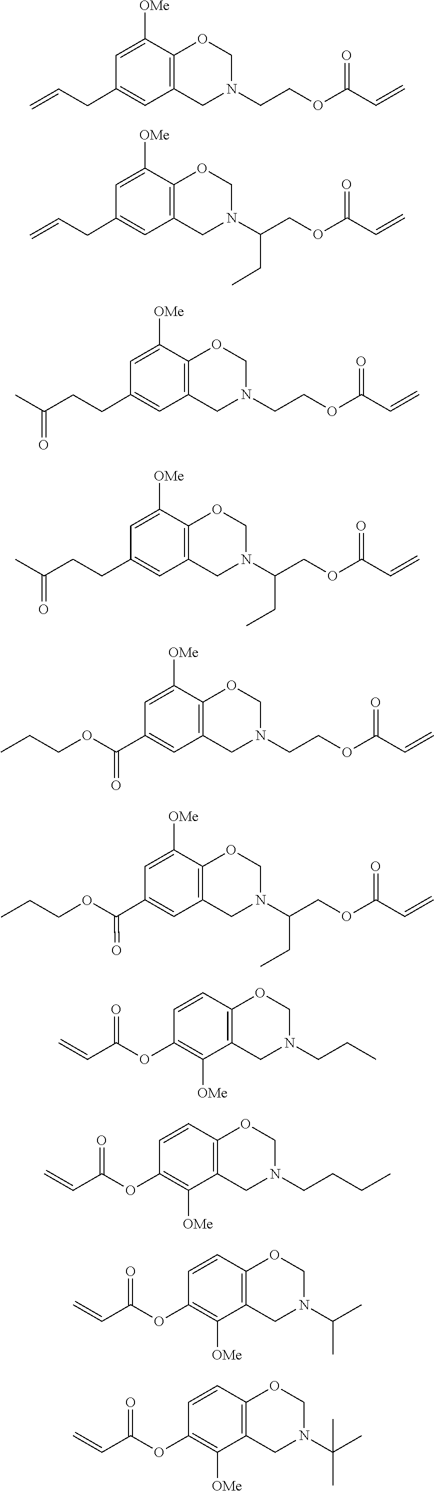

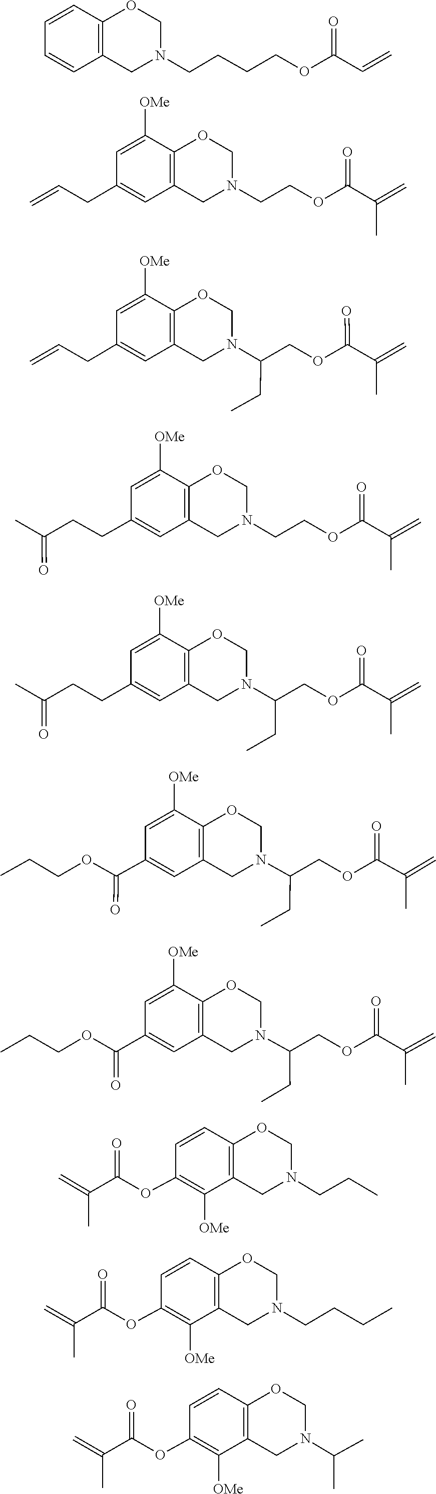

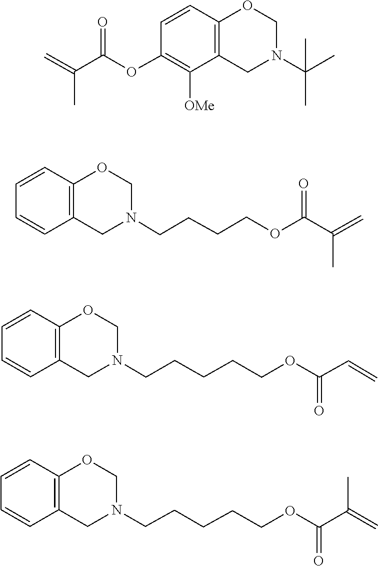 [SMILES: C=C(C)C(=O)Oc1ccc2c(c1OC)CN(C(C)(C)C)CO2 ; C=C(C)C(=O)OCCCCN1COc2ccccc2C1 ; C=CC(=O)OCCCCCN1COc2ccccc2C1 ; C=C(C)C(=O)OCCCCCN1COc2ccccc2C1]

9. A formulation for additive manufacturing, comprising:
one or more monomers according to formula I or formula II as described in claim 4; and
a photoinitiator;

10. (canceled)

11. The formulation according to claim 9, wherein the formulation further comprises one or more of an antioxidant, a stabiliser, a colourant, a diluent, a flame retardant, a plasticizer, a photoabsorber, a photoinhibitor, and a filler.

12. The formulation according to claim 9, wherein the formulation further comprises an acrylate monomer that does not have the formula I or the formula II.

13. The formulation according to claim 9, wherein the monomer according to formula I or formula II is a monomer according to formula Ia or formula IIa: 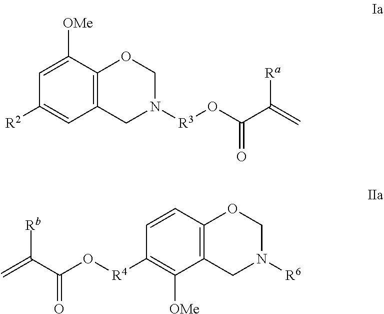
where:
R² is selected from H, CO₂R⁷. linear or branched C₁ to C₅ alkyl, linear or branched C₂ to C₅ alkenyl, where the latter two substituents are unsubstituted or substituted by one or more substituents selected from OR^{8a}, ═O, and C₁ to C₃ alkyl;
R³ is a linear or branched C₂ to C₅ alkyl;
R⁴ is a linear or branched C₁ to C₅ alkyl;
R⁶ is a linear or branched C₁ to C₅ alkyl, which is unsubstituted or substituted by one or more substituents selected from OR^{8a}, ═O, and C₁ to C₃ alkyl;
R⁷ is a linear or branched C₁ to C₅ alkyl; and
R^{a} and R^{b} are selected from H and CH₃.

14. The formulation according to claim 13, wherein:
R² is selected from H, CO₂R⁷. linear or branched C₂ to C₄ alkyl, linear or branched C₂ to C₃ alkenyl, where the latter two substituents are unsubstituted or substituted by one or more substituents selected from ═O, and C₁ to C₃ alkyl;
R³ is a linear or branched C₂ to C₅ alkyl;
R⁴ is C₁ alkyl;
R⁶ is a linear or branched C₂ to C₄ alkyl, which is unsubstituted or substituted by one or more substituents selected from C₁ to C₃ alkyl;
R⁷ is a linear or branched C₂ to C₄ alkyl.

15. The formulation according to claim 9, wherein the monomer is selected from the list:

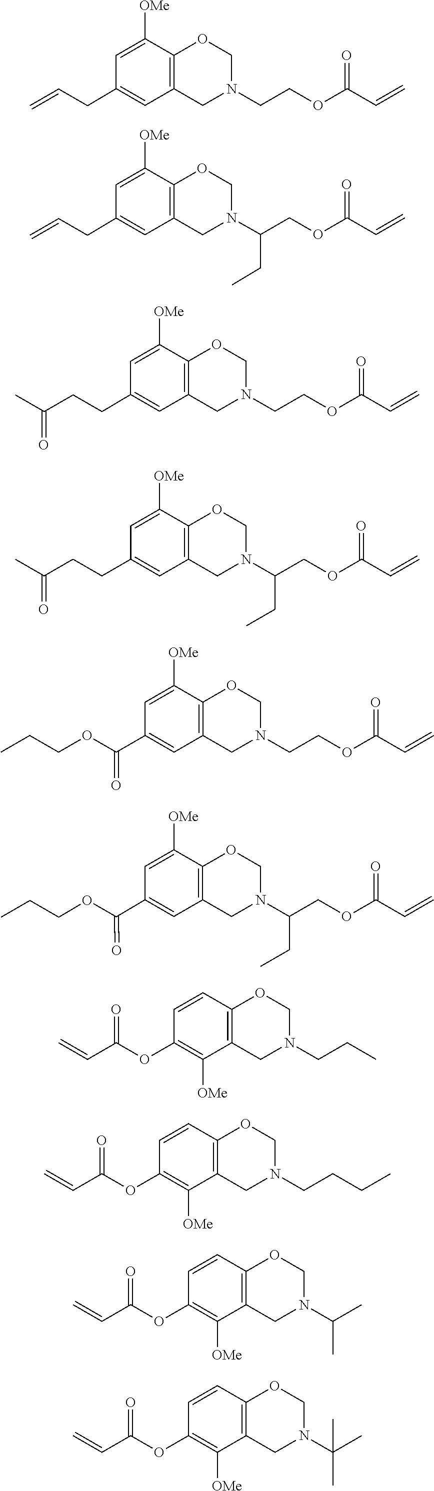

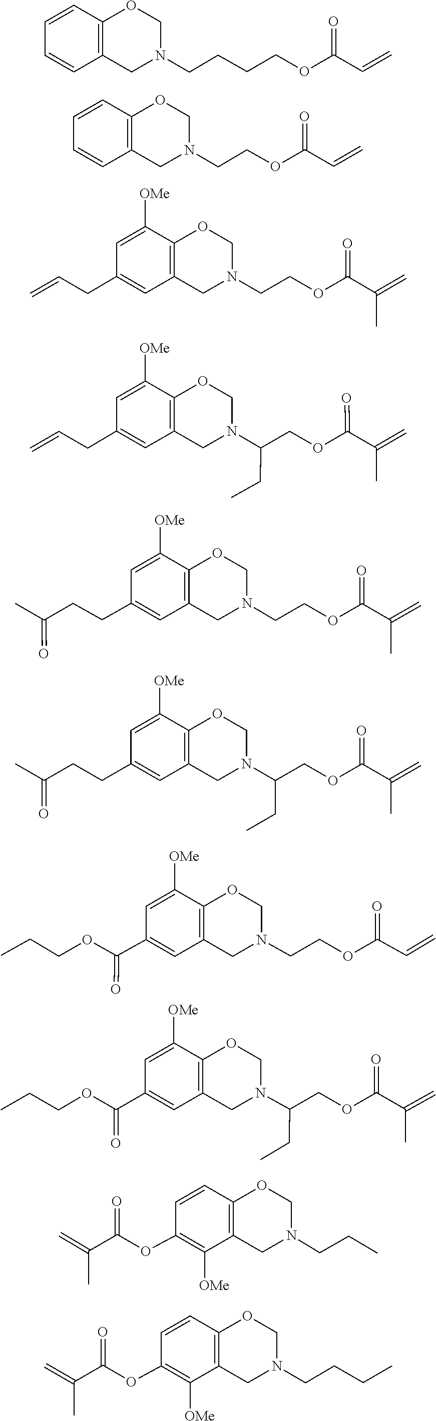

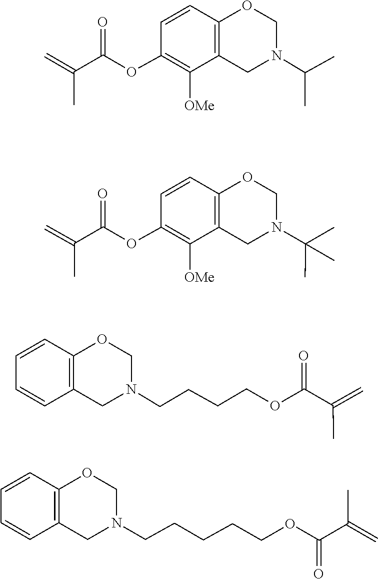

16. The formulation according to claim 9, wherein the monomer is selected from the list:

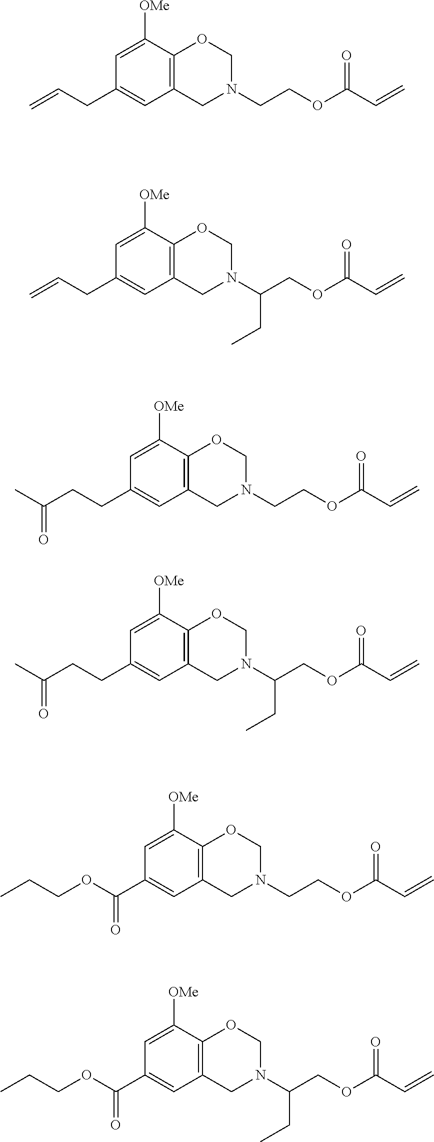

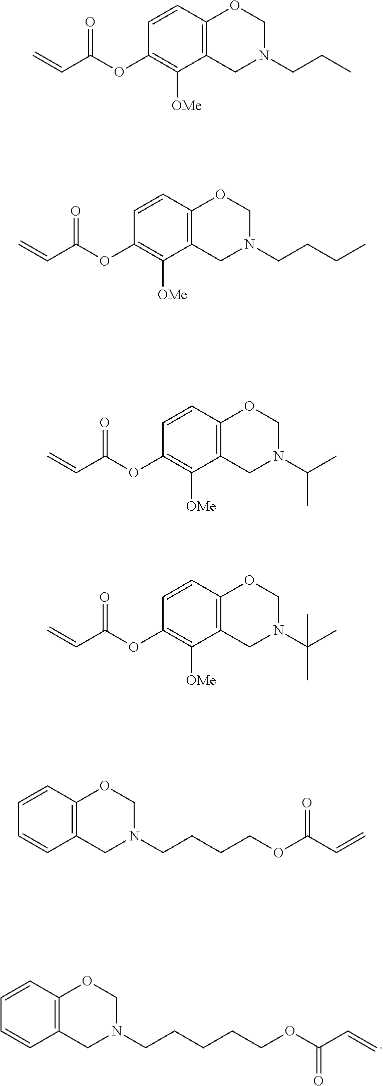

17. The formulation according to claim 9, wherein the formulation comprises the following monomers:

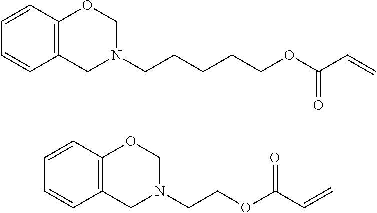 [SMILES: C=CC(=O)OCCCCCN1COc2ccccc2C1 ; C=CC(=O)OCCN1COc2ccccc2C1]

18. A method of providing an intermediate product by additive manufacturing, the method comprising the steps of:
(a) 3D-printing an object one layer at a time according to a product design using a printing resin, where each layer is subjected to ultraviolet light for a first period of time before each further layer is added; and
(b) stopping the 3D-printing once the product design has been generated, thereby providing an intermediate product, wherein:
the printing resin is a monomer according to claim 4; and
the product is capable of being cured further using a thermal curing step.

19. (canceled)

20. (canceled)

21. A method of providing a final product by additive manufacturing, the method comprising the steps of:
(a) a providing an intermediate product formed by an additive manufacturing process; and
(b) subjecting the intermediate product to a thermal curing step using a suitable temperature for a period of time, wherein
the intermediate product is formed from polymeric matrix material, where the polymeric matrix material comprises repeating units derived from a monomer according to claim 4.

22. (canceled)

23. (canceled)

24. (canceled)

25. A method of providing an intermediate product by additive manufacturing, the method comprising the steps of:
(a) 3D-printing an object one layer at a time according to a product design using a printing resin, where each layer is subjected to ultraviolet light for a first period of time before each further layer is added; and
(b) stopping the 3D-printing once the product design has been generated, thereby providing an intermediate product, wherein:
the printing resin is a formulation according to claim 9; and
the product is capable of being cured further using a thermal curing step.
